(19) United States
(12) Patent Application Publication
(10) Pub. No.: US 2023/0097603 A1
(43) Pub. Date: Mar. 30, 2023

(54) NICOTINAMIDE MONONUCLEOTIDE DERIVATIVES FOR THE TREATMENT OF ARRHYTHMIA

(71) Applicant: NUVAMID SA, Epalinges (CH)

(72) Inventors: Laurent GARÇON, Epalinges (CH); Guillaume BERMOND, Epalinges (CH)

(73) Assignee: NUVAMID SA, Epalinges (CH)

(21) Appl. No.: 17/908,337

(22) PCT Filed: Mar. 5, 2021
(86) PCT No.: PCT/EP2021/055676

§ 371 Date: Aug. 31, 2022

(30) Foreign Application Priority Data
Mar. 6, 2020 (FR) FR2002267

(51) Int. Cl. A61K 31/7084; A61K 31/706; A61P 9/06

(52) U.S. Cl. CPC A61K 31/7084; A61K 31/706; A61P 9/06

(57) ABSTRACT

A method for treating arrythmia in subject in need thereof, which includes administering to the subject a therapeutically effective amount of the compound of formula (I) or a pharmaceutically acceptable salt and/or solvate thereof;

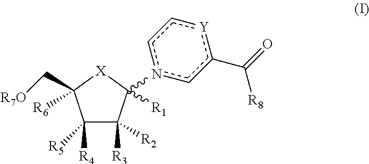

in which X, R₁, R₂, R₃, R₄, R₅, R₆, R₇, R₈, Y, [symbol] and [symbol] are as described in the claims.

### FIELD OF THE INVENTION

The present invention relates to a compound of formula (I) or a pharmaceutically acceptable salt and/or solvate thereof for use in the treatment and/or prevention of arrhythmia.

### PRIOR ART

Sudden cardiac arrest is characterised by a sudden loss of cardiac functions, breathing and consciousness, usually caused by an interruption in the normal electrical activity of the heart.

The majority of deaths from sudden cardiac arrest (SCD) are caused by a fatal cardiac arrhythmia and a loss of pumping capability of the heart, the most frequent being ventricular tachycardia (VT) and ventricular fibrillation (VF). As a consequence, the heart may suddenly stop (sudden cardiac death) and require immediate emergency resuscitation.

Ventricular tachycardia and ventricular fibrillation are characterised by a rapid and chaotic rhythm originating from the lower chambers of the heart, preventing the heart from pumping blood to the rest of the body.

Arrhythmic conditions are generally associated with systematic oxidative stress and cardiac stress caused by reactive oxygen species (‘Radical Oxygen Species’ or ‘ROS’), which leads to a disequilibrium between the oxidised (NAD+) and reduced (NADH) forms of nicotinamide adenine dinucleotide NAD and a depletion of the main intracellular antioxidants.

High cellular levels of ROS can cause alterations in the cardiac sodium channel (Nav1.5). It has been shown that raising the intracellular level of NADH can acutely reduce the Na+ current (iNa) and to a sufficiently large extent to be clinically significant. Indeed, it appears that the oxidised form of nicotinamide adenine dinucleotide (NAD+) increases the current of the sodium channel and the level of the sodium channel, whereas NADH, the reduced form, reduces the current of the sodium channel and the level of the sodium channel.

Currently, the treatment of arrhythmia essentially involves drug treatment aimed at slowing the too rapid heart rate with beta-blockers such as atenolol, metoprolol or even calcium inhibitors such as verapamil. Drugs derived from digitalis, such as digoxin for example, are also commonly used for the treatment of cardiac arrhythmia. On the other hand, anti-arrhythmic agents can be prescribed with the aim of recovering regular heartbeats. These are, for example, amiodarone, propafenone or sotalol.

In certain cases, cardiac arrhythmia will be treated by ablation of the abnormal tissue at radio frequencies, this method enabling the heart rhythm to be re-established.

However, the effectiveness of these drugs varies according to the patient and the causes of the observed arrhythmia. Some of these drugs are also used to reduce auricular or ventricular fibrillation (“drug-based cardioversion”).

Because their use is delicate, drugs for heart rhythm disorders are only prescribed when these disorders cause significant daily discomfort or if they risk serious consequences.

The prevention of heart rhythm disorders is based on general hygiene measures recommended for the health of the heart, such as a balanced diet, stopping smoking, moderate alcohol consumption and regular physical exercise.

Hence, there is a need for effective and well tolerated treatments or prevention means for a large number of patients, whatever the type of cardiac arrhythmia.

The aim of this invention is to propose an alternative to current treatments by providing nicotinamide mononucleotide derivatives for the treatment and prevention of arrhythmia.

The Applicant has observed that the derivatives of nicotinamide mononucleotide according to the invention are well-tolerated and can reduce the frequency of arrhythmias in a reperfused ischaemic rat model.

### SUMMARY

The present invention relates to a compound of formula (I)

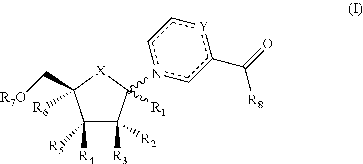

or a pharmaceutically acceptable salt and/or solvate thereof, wherein:
X is selected from O, CH₂, S, Se, CHF, CF₂ and C═CH₂;
R₁ is selected from H, azido, cyano, C₁-C₈ alkyl, C₁-C₈ thio-alkyl, C₁-C₈ heteroalkyl and OR; wherein R is selected from H and C₁-C₈ alkyl;
R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl,
C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
R₆ is selected from H, azido, cyano, C₁-C₈ alkyl, C₁-C₈ thio-alkyl, C₁-C₈ heteroalkyl and OR; wherein R is selected from H and C₁-C₈ alkyl;
R₇ is selected from P(O)R₉R₁₀, P(S)R₉R₁₀ and

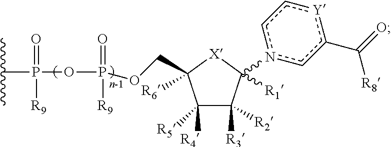

wherein
R₉ and R₁₀ are selected, independently of one another, from OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, (C₅-C₁₂)-aryl-(C₁-C₈)-alkyl, (C₁-C₈)-alkyl-(C₅-C₁₂)-aryl, (C₁-C₈)-heteroalkyl, (C₃-C₈)-heterocycloalkyl, (C₅-C₁₂)-heteroaryl and NHCR_{α}R_{α′}—C(O)R₁₂; wherein:
   R₁₁ is selected from C₁-C₁₀ alkyl, C₃-C₁ cycloalkyl, C₅-C₁₂ aryl, (C₁-C₁₀)-alkyl-(C₅-C₁₂)-aryl, C₅-C₁₂ substituted aryl, C₁-C₁₀ heteroalkyl, C₁-C₁₀ haloalkyl, —(CH₂)ₘC(O)(C₁-C₁₅)-alkyl, —(CH₂)ₘOC(O)(C₁- C₁₅)-alkyl, —(CH₂)ₘOC(O)O(C₁-C₁₅)-alkyl, —(CH₂)ₘSC(O)(C₁-C₁₅)-alkyl, —(CH₂)ₘC(O)O(C₁-C₁₅)-alkyl, —(CH₂)ₘC(O)O(C₁-C₁₅)-alkyl-aryl; wherein m is an integer selected from 1 to 8; and P(O)(OH)OP(O)(OH)₂; an internal or external counter-ion;
   R₁₂ is selected from hydrogen, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₂ aryl, (C₁-C₄)-alkyl-(C₅-C₁₂)-aryl and C₅-C₁₂ heteroaryl; wherein said aryl or heteroaryl groups are optionally substituted by one or two groups selected from halogen, trifluoromethyl, C₁-C₆ alkyl, C₁-C₆ alkoxy and cyano;
   R₁₃ and R₁₄ are selected independently from H, C₁-C₈ alkyl and (C₁-C₈)-alkyl-(C₅-C₁₂)-aryl;
   R_{α} and R_{α′} selected independently from hydrogen, C₁-C₁₀ alkyl, C₂-C₁₀ alkenyl, C₂-C₁₀ alkynyl, C₃-C₁₀ cycloalkyl, C₁-C₁₀ thio-alkyl, C₁-C₁₀ hydroxylalkyl, (C₁-C₁₀)-alkyl-(C₅-C₁₂)-aryl, C₅-C₁₂ aryl, —(CH₂)₃NHC(═NH)NH₂, (1H-indol-3-yl)-methyl, (1H-imidazol-4-yl)-methyl and a side chain selected from a proteinogenic or non-proteinogenic amino acid; wherein said aryl groups are optionally substituted by a group selected from hydroxyl, C₁-C₁₀ alkyl, C₁-C₆ alkoxy, halogen nitro and cyano;
or R₉ and R₁₀, with the phosphorus atoms to which they are bonded, form a 6-member-ring, wherein —R₉-R₁₀— represents —CH₂—CH₂—CHR— or —O—CH₂—CH₂—CHR—O—; wherein R is selected from hydrogen, C₅-C₆ aryl and C₅-C₆ heteroaryl; wherein said aryl or heteroaryl groups are optionally substituted by one or two groups selected from halogen, trifluoromethyl, C₁-C₆ alkyl, C₁-C₆ alkoxy and cyano;
X′ is selected from O, CH₂, S, Se, CHF, CF₂ and C═CH₂;
R_{1′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{2′}, R_{3′}, R_{4′} and R_{5′} are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein RA is a side chain selected from a proteinogenic amino acid;
R_{6′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{8′} is selected from H, OR, NHR_{15′}, NR_{15′}R_{16′}, NH—NHR_{15′}, SH, CN, N₃ and halogen; wherein R_{15′} and R_{16′} are selected, independently of one another, from H, C₁-C₈ alkyl and C₁-C₈ alkyl-aryl;
Y′ selected from CH, CH₂, C(CH₃)₂ and CCH₃;
n is an integer selected from 1 to 3;
[symbol] represents a single or a double bond according to Y′; and
[symbol] presents the alpha or beta anomer according to the position of R_{1′};
R₈ is selected from H, OR, NHR₁₅, NR₁₅R₁₆, NH—NHR₁₅, SH, CN, N₃ and halogen; wherein R is selected from H and C₁-C₈ alkyl, and R₁₅ and R₁₆ are selected, independently of one another, from H, C₁-C₈ alkyl and C₁-C₈ alkyl-aryl and —CHR_{AA}CO₂H wherein R_{AA} is a side chain selected from a proteinogenic or non-proteinogenic amino acid;
Y is selected from CH, CH₂, C(CH₃)₂ and CCH₃;
[symbol] represents a single or a double bond according to Y; and
[symbol] presents the alpha or beta anomer according to the position of R₁,
   for use thereof in the treatment of arrhythmia.

In an embodiment, X represents oxygen.

In an embodiment, R₁ and R₆ each represent hydrogen.

In an embodiment, R₂, R₃, R₄ and R₅ each represent, independently of one another, hydrogen or an OH.

In an embodiment, Y represents CH.

In an embodiment, Y represents CH₂.

In an embodiment, R₇ represents P(O)R₉R₁₀ or

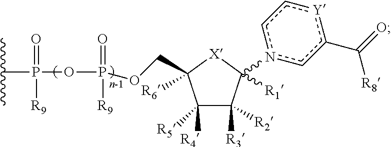

wherein R₉ and R₁₀ are as defined in formula (I) and
X′ is oxygen;
R_{1′} and R_{6′} each represent hydrogen;
R_{2′}, R_{3′}, R_{4′} and R_{5′} are independently selected from hydrogen and OH;
R_{8′} is NH₂;
Y′ is selected from CH and CH₂;
n is equal to 2;
[symbol] represents a single or a double bond according to Y′; and
[symbol] represents the alpha or beta anomer according to the position de R_{1′}.

In an embodiment, R₇ represents P(O)(OH)₂.

In an embodiment, the compound of the invention is selected from:

TABLE 1

| | |
|---|---|
| 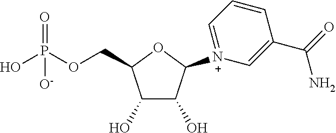 [SMILES: NC(=O)c1ccc[n+]([C@@H]2O[C@H](COP(=O)([O-])O)[C@@H](O)[C@H]2O)c1] | I-C |
| 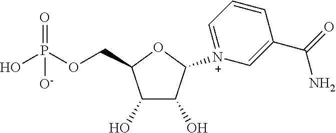 [SMILES: NC(=O)c1ccc[n+]([C@H]2O[C@H](COP(=O)([O-])O)[C@@H](O)[C@H]2O)c1] | I-D |
| 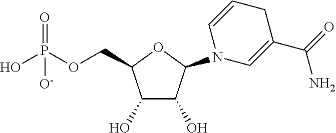 [SMILES: NC(=O)C1=CN([C@@H]2O[C@H](COP(=O)([O-])O)[C@@H](O)[C@H]2O)C=CC1] | I-G |
| 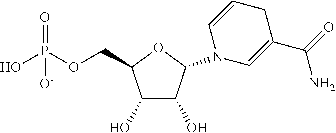 [SMILES: NC(=O)C1=CN([C@H]2O[C@H](COP(=O)([O-])O)[C@@H](O)[C@H]2O)C=CC1] | I-H |
| 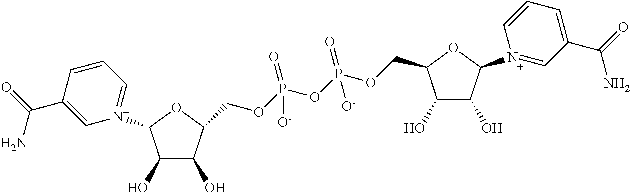 [SMILES: NC(=O)c1ccc[n+]([C@@H]2O[C@H](COP(=O)([O-])OP(=O)([O-])OC[C@H]3O[C@@H]([n+]4cccc(C(N)=O)c4)[C@H](O)[C@@H]3O)[C@@H](O)[C@H]2O)c1] | I-I |
| 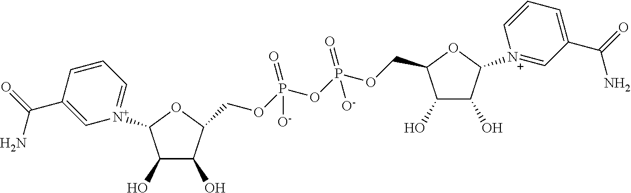 [SMILES: NC(=O)c1ccc[n+]([C@@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] | I-J |
| 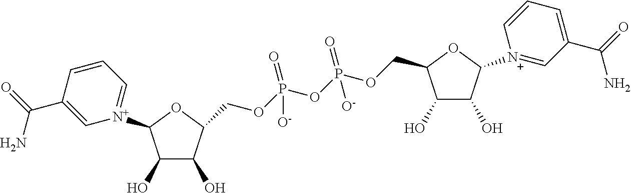 [SMILES: NC(=O)c1ccc[n+]([C@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] | I-K |
| 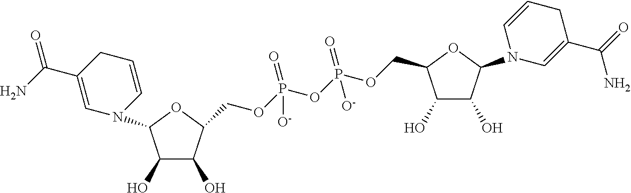 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] | I-L |
| 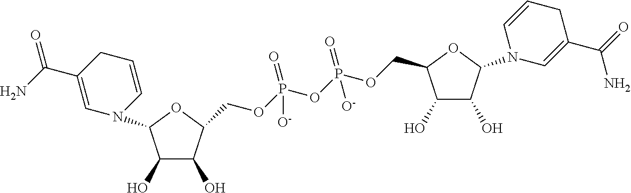 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] | I-M |
| 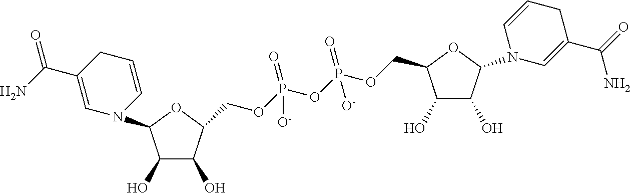 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] | I-N |

or a pharmaceutically acceptable salt and/or solvate thereof.

In an embodiment, the type of arrhythmia is selected from bradycardia, tachycardia, auricular fibrillation, ventricular tachycardia and/or ventricular fibrillation.

### Definitions

In the present invention the following terms have the following meaning.

Unless otherwise indicated, the nomenclature of the substituents which are not explicitly defined in the present invention is obtained by naming the terminal part of the functionality followed by the adjacent functionality towards the point of attachment.
“Alkyl” by itself or as part of another substituent, means a hydrocarbyl radical of formula CnH2n+1 in which n is a number greater than or equal to 1. In general, the alkyl groups of this invention comprise 1 to 12 carbon atoms, preferably 1 to 8 carbon atoms, more preferably 1 to 6 carbon atoms, even more preferably 1 to 2 carbon atoms. The alkyl groups can be linear or branched and can be substituted as indicated in the present invention. Suitable alkyl groups include methyl, ethyl, n-propyl, i-propyl, n-butyl, i-butyl, s-butyl and t-butyl, pentyl and its isomers (for example, n-pentyl, iso-pentyl), hexyl and its isomers (for example, n-hexyl, iso-hexyl), heptyl and its isomers (for example, n-heptyl, iso-heptyl), octyl and its isomers (for example n-octyl, iso-octyl), nonyl and its isomers (for example, n-nonyl, iso-nonyl), decyl and its isomers (for example n-decyl, iso-decyl), undecyl and its isomers, dodecyl and its isomers. The preferred alkyl groups are the following: methyl, ethyl, n-propyl, i-propyl, n-butyl, i-butyl, s-butyl and t-butyl. The Cx-Cy-alkyls mean the alkyl groups which comprise from x to y carbon atoms.
“Alkenyl” by itself or as part of another substituent, means an unsaturated hydrocarbyl group, which may be linear or branched, comprising one or more carbon-carbon double bonds. Suitable alkenyl groups comprise between 2 and 12 carbon atoms, preferably between 2 and 8 carbon atoms, even more preferably between 2 and 6 carbon atoms. Non-limiting examples of alkenyl groups include ethenyl, 2-propenyl, 2-butenyl, 3-butenyl, 2-pentenyl and its isomers, 2-hexenyl and its isomers, 2,4-pentadienyl.
“Alkynyl” by itself or as part of another substituent, means a class of unsaturated monovalent groups, in which the unsaturation results from the presence of one or more carbon-carbon triple bonds. The alkynyl groups generally, and preferably, have the same number of carbon atoms as described above for the alkenyl groups. Non-limiting examples of alkynyl groups include ethynyl, 2-propynyl, 2-butynyl, 3-butynyl, 2-pentynyl and its isomers, 2-hexynyl and its isomers.
“Alkoxy” means an alkyl group as defined above, which is attached to another part by an oxygen atom. Examples of alkoxy groups include methoxy, isopropoxy, ethoxy, tert-butoxy and other groups. The alkoxy groups may optionally be substituted by one or more substituents. The alkoxy groups included in the compounds of this invention can optionally be substituted by a solubilising group.
“Aryl”, as it is used here, means an aromatic, polyunsaturated hydrocarbyl group having a single ring (for example phenyl) or a plurality of aromatic rings that are fused together (for example naphtyl) or covalently bonded, generally containing 5 to 12 atoms, preferably 6 to 10, at least one ring of which is aromatic. The aromatic ring can optionally comprise one or two additional rings (cycloalkyl, heterocyclyl or heteroaryl) which are fused thereto. The aryl is also intended to include the partially hydrogenated derivatives of the carbocyclic systems listed here. The aryl examples comprise phenyl, biphenyl, biphenylenyl, the 5- or 6-tetralinyl, naphtalene-1- or -2-yl, 4-, 5-, 6 or 7-indenyl, 1-2-, 3-, 4- or 5-acenaphtylenyl, 3-, 4- or 5-acenaphtenyl, 1- or 2-pentalenyl, 4- or 5-indanyl, 5-, 6-, 7- or 8-tetrahydronaphtyl, 1,2,3,4-tetrahydronaphtyl, 1,4-dihydronaphtyl, 1-, 2-, 3-, 4- or 5-pyrenyl.
“Alkylaryl” means an aryl group substituted by an alkyl group.
“Amino acid” means an alpha-aminated carboxylic acid, in other words a molecule comprising a functional carboxylic acid group and a functional amine group in the alpha position of the carboxylic acid group, for example a proteinogenic amino acid or a non-proteinogenic amino acid such as 2-aminoisobutyric acid.
“Proteinogenic amino acid” means an amino acid which is incorporated in the proteins during the translation of the messenger RNA by the ribosomes in living organisms, in other words alanine (ALA), arginine (ARG), asparagine (ASN), aspartate (ASP), cysteine (CYS), glutamate (glutamic acid) (GLU), glutamine (GLN), glycine (GLY), histidine (HIS), isoleucine (ILE), leucine (LEU), lysine (LYS), methionine (MET), phenylalanine (PHE), proline (PRO), pyrrolysine (PYL), selenocysteine (SEL), serine (SER), threonine (THR), tryptophan (TRP), tyrosine (TYR) or valine (VAL).
“Non-proteinogenic amino acid” means an amino acid which is not naturally encoded or is not found in the genetic code of a living organism. Non-limiting examples of non-proteinogenic amino acids include ornithine, citrulline, argininosuccinate, homoserine, homocysteine, cysteine-sulfinic acid, 2-aminomuconic acid, δ-aminolevulinic acid, β-alanine, cystathionine, γ-aminobutyrate, DOPA, 5-hydroxytryptophan, D-serine, ibotenic acid, α-aminobutyrate, 2-aminoisobutyrate, D-leucine, D-valine, D-alanine or D-glutamate.
“cycloalkyl” by itself or as part of another substituent means a cyclical alkyl, alkenyl or alkynyl group, in other words a saturated or unsaturated, monovalent hydrocarbyl group, having 1 or 2 cyclic structures. Cycloalkyl includes monocyclic or bicyclic hydrocarbyl groups. The cycloalkyl groups may comprise 3 carbon atoms or more in the ring and generally, according to this invention, comprise 3 to 10, preferably 3 to 8, yet more preferably 3 to 6 carbon atoms. Non-limiting examples of cycloalkyl groups include cyclopropyl, cyclobutyl, cyclopentyl and cyclohexyl; cyclopropyl being particularly preferred.
The term “pharmaceutically acceptable excipient” refers to an inert carrier or support used as a solvent or diluent, in which the pharmaceutically active agent it is formulated and/or administered, and which does not produce an undesirable, allergic or other reaction when it is administered to an animal, preferably a human being. This includes all solvents, dispersion media, coatings, antibacterial and antifungal agents, isotonic agents, absorption retardants and other similar ingredients. For human administration, the preparations must meet the standards of sterility, general safety and purity, as required by the regulating offices, such as the FDA or EMA, for example. Within the meaning of the invention, “pharmaceutically acceptable excipients” includes all pharmaceutically acceptable excipients as well as all pharmaceutically acceptable supports, diluents, and/or additives.
“Halogen” or “halo” means fluoro, chloro, bromo or iodo. The preferred halo groups are fluoro and chloro.
“Haloalkyl” alone or in combination, means an alkyl radical having the meaning as defined above, wherein one or more hydrogen atoms are replaced by a halogen as defined above. Examples of such halogenoalkyl radicals include chloromethyl, 1-bromoethyl, fluoromethyl, difluoromethyl, trifluoromethyl, the 1,1,1-trifluoroethyl and similar radicals. Cx-Cy-haloalkyl and Cx-Cy-alkyl mean alkyl groups which comprise from x to y carbon atoms. The preferred halogenoalkyl groups are difluoromethyl and trifluoromethyl.
“Heteroalkyl” means an alkyl group as defined above, in which one or more carbon atoms are replaced by a heteroatom selected from the atoms of oxygen, nitrogen and sulfur. In the heteroalkyl groups, the heteroatoms are only bonded to carbon atoms along the alkyl chain, in other words each heteroatom is separated from any other heteroatom by at least one carbon atom. However, the heteroatoms of nitrogen and sulfur can optionally be oxidised and the heteroatoms of nitrogen can optionally be quaternised. A heteroalkyl is bonded to another group or to another molecule only via a carbon atom, in other words the bond atom is not selected from the heteroatoms included in the heteroalkyl group.
“Heteroaryl” by itself or as part of another substituent means aromatic rings having 5 to 12 carbon atoms or systems containing 1 to 2 rings that are fused or covalently bonded, typically containing 5 to 6 atoms; at least one of the rings being aromatic; wherein one or more carbon atoms in one or more rings is replaced by oxygen, nitrogen and/or sulfur atoms; the heteroatoms of nitrogen and sulfur can optionally be oxidised and the heteroatoms nitrogen can optionally be quaternised. Such rings can be fused with an aryl, cycloalkyl, heteroaryl or heterocyclyl ring. Non-limiting examples of heteroaryls include: furanyl, thiophenyl, pyrazolyl, imidazolyl, oxazolyl, isoxazolyl, thiazolyl, isothiazolyl, triazolyl, oxadiazolyl, thiadiazolyl, tetrazolylr, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyel, 2,1,3-benzothiadiazolyl, thidnopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl. When at least one carbon atom in a cycloalkyl group is replaced by a heteroatom, the resulting ring is called “heterocycloalkyl” or “heterocyclyl”.
“Heterocyclyl”, “heterocycloalkyl” or “heterocyclo” by itself or as part of another substituent means fully saturated or partially unsaturated, cyclic, non-aromatic groups, (for example, monocycle with 3 to 7 members, bicycle with 7 to 11 members, or comprising a total of 3 to 10 atoms in the ring) which have at least one heteroatom in at least one ring containing carbon atoms. Each ring of the heterocyclyl group comprising a heteroatom can have 1, 2, 3 or 4 heteroatoms selected from nitrogen, oxygen and/or sulfur atoms, nitrogen and sulfur heteroatoms can optionally be oxidised and the nitrogen heteroatoms can optionally be quaternised. Each carbon atom of the heterocycle can be substituted by oxo (for example piperidone, pyrrolidinone). The heterocyclic group can be attached to any carbon atom or heteroatom of the ring or of the cyclic system, when the valence allows it. The rings of the multi-cyclic heterocycles can be fused, bridged and/or joined by one or more spiro atoms. Non-limiting examples of heterocyclic groups include oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylulfoxide, thiomorpholin-4-ylsulfonyl, 1,3-dioxolanyl, 1,4-oxathianyl, 1H-pyrrolizinyl, tetrahydro-1,1-dioxothiophenyl, N-formylpiperazinyl, and morpholin-4-yl.
The “pharmaceutically acceptable salts” comprise the acid and base addition salts of these salts. Suitable acid addition salts are formed from acids which form non-toxic salts. This includes, for example, acetate, adipate, aspartate, benzoate, besylate, bicarbonate/carbonate, bisulfate/sulfate, borate, camsylate, citrate, cyclamate, edisylate, esylate, formiate, fumarate, gluceptate, gluconate, glucuronate, hexafluorophosphate, hibenzate, chlorhydrate/chloride, bromhydrate/bromide, hydroiodide/iodide, isethionate, lactate, malate, maleate, malonate, mesylate, methylsulfate, naphtylate, 2-napsylate, nicotinate, nitrate, orotate, oxalate, palmitate, pamoate, phosphate/hydrogenophosphate/dihydrogenophosphate, pyroglutamate, saccharate, stearate, succinate, tannate, tartrate, tosylate, trifluoroacetate and xinofoate salts. Suitable basic salts are formed from bases which form non-toxic salts. These include, for example the salts of aluminium, arginine, benzathine, calcium, chlorine, diethylamine, diolamine, glycine, lysine, magnesium, meglumine, olamine, potassium, sodium, tromethamine, 2-(diethylamino)ethanol, ehanolamine, morpholine, 4-(2-hydroxyethyl)morpholine and zinc. Acid and base hemisalts can also be formed, for example, hemisulfates and chemical calcium salts. The preferred pharmaceutically acceptable salts are the chlorhydrate/chloride, bromide/hydrobromide, bisulfate/sulfate, nitrate, citrate and acetate.

The pharmaceutically acceptable salts can be prepared by one or more of these methods:
(i) by reacting the compound with the desired acid;
(ii) by reacting the compound with the desired base;
(iii) by removing a protective labile group in an acid or base medium of a suitable precursor of the compound or by opening the ring of a suitable cyclic precursor, for example a lactone or a lactam, using the desired acid; or
iv) by transforming one salt of the compound into another, by reaction with a suitable acid or by means of a suitable ion-exchange column.

All these reactions are generally carried out in solution. The salt can precipitate from the solution and be collected by filtration or can be recovered by evaporation of the solvent. The degree of ionisation of the salt can vary from completely ionised to almost unionised.
“Pharmaceutically acceptable” means approved or able to be approved by a regulating body or included in a known pharmacopoeia for use in animals, and more preferably in humans. It may be a substance that is not biologically or otherwise undesirable, in other words the substance can be administered to an individual without causing undesirable biological effects or deleterious interactions with one of the components of the composition in which it is contained.
“Solvate” is used here to describe a molecular complex comprising the compound of the invention and one or more molecules of pharmaceutically acceptable solvent, for example ethanol.
The term “substituent” or “substituted” means that a hydrogen radical on a compound or a group is replaced by any desired group which is substantially stable under the reaction conditions in an unprotected form or when it is protected by a protective group. The examples of preferred substituents are those found in the compounds and embodiments presented here, as well as the halogeno, alkyl or aryl groups as defined above, hydroxyl, alkoxy groups as defined above, nitro, thiol, heterocycloalkyl, heteroaryl, cyano, cycloalkyl groups as defined above, as well as a solubilising group, —NRR′, —NR—CO—R′, —CONRR′, —SO2NRR′, where R and R′ are each independently selected from hydrogen and the alkyl, cycloalkyl, aryl, heterocycloalkyl or heteroaryl groups as defined above.
The asymmetric carbon bonds can be represented here by using a solid triangle ( [symbol] ), a dotted triangle ( [symbol] ) or a zigzag line ( [symbol] ).
The term “administration”, or a variant of this term (for example, “to administer”), means delivering the active agent or active substance, alone or in a pharmaceutically acceptable composition, to the patient for whom the symptom or disease must be treated or prevented.
“Arrhythmia” relates to heart rhythm disorders, in other words a disturbance in the normal rhythm of the heart, which can be benign or require a suitable treatment. Depending on the speed of the heartbeat, a person skilled in the art would be able to determine the type of arrhythmia. In an embodiment, it is a bradycardia. In an embodiment, it is a tachycardia. In an embodiment, it is an auricular fibrillation. In an embodiment, it is a ventricular tachycardia. In an embodiment, it is a ventricular fibrillation.
The term “subject” refers to a mammal, preferably a human. According to the present invention, a subject is a mammal, preferably a human, suffering from arrhythmia. According to an embodiment, the subject is a “patient”, i.e. a mammal, preferably a human, who is waiting to receive, or who is receiving medical care or who was/is/will be the subject of a medical procedure, or who is monitored for the development of an arrhythmia.
The term “human” refers to a subject of both sexes and at any stage of development (in other words newborn, infant, juvenile, adolescent, adult).
The term “therapeutically effective quantity” (or more simply an “effective quantity”) as used here refers to the quantity of active agent or active ingredient which is targeted, without causing significant negative or undesirable side effects for the subject needing treatment, prevention, reduction, relief or slowing (attenuation) of one or more symptoms or manifestations of arrhythmia.
The terms “to treat” or “treatment”, as used here, mean a therapeutic treatment, a prophylactic (or preventive) treatment, or both a therapeutic treatment and a prophylactic (or preventive) treatment, wherein the aim is to prevent, reduce, relieve and/or slow (attenuate) one or more symptoms or manifestations of arrhythmia, in a subject having need thereof. The symptoms and manifestations of arrhythmia comprise, but are not limited to, modification of the heart rhythm, shortness of breath and fatigue. In an embodiment, “to treat” or “treatment” refers to a therapeutic treatment. In another embodiment, “to treat” or “treatment” refers to a prophylactic or preventive treatment. In yet another embodiment, “to treat” or “treatment” means both a prophylactic (or preventive) treatment and a therapeutic treatment. In an embodiment, the aim of the treatment according to the present invention is to cause at least one of the following elements:
   (a) improvement in the clinical condition of the patient, in particular a reduction or disappearance of the palpitations, shortness of breath and/or fatigue;
   (b) normalisation of the heart rhythm.

### DETAILED DESCRIPTION

The present invention therefore relates to the use of nicotinamide mononucleotide derivatives for the treatment of arrhythmia.

Compounds for Treating Arrhythmia.

The present invention relates to a compound of formula (I)×

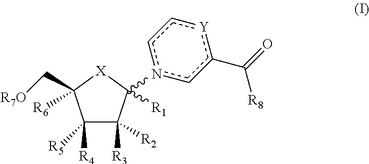

or a pharmaceutically acceptable salt and/or solvate thereof, wherein:
   X is selected from O, CH₂, S, Se, CHF, CF₂ and C═CH₂;
   R₁ is selected from H, azido, cyano, C₁-C₈ alkyl, C₁-C₈ thio-alkyl, C₁-C₈ heteroalkyl and OR; wherein R is selected from H and C₁-C₈ alkyl;
   R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
   R₆ is selected from H, azido, cyano, C₁-C₈ alkyl, C₁-C₈ thio-alkyl, C₁-C₈ heteroalkyl and OR; wherein R is selected from H and C₁-C₈ alkyl;
   R₇ is selected from P(O)R₉R₁₀, P(S)R₉R₁₀ and

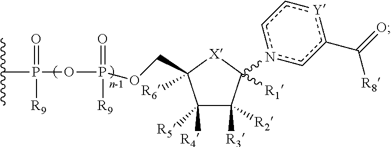

R₉ and R₁₀ are selected, independently of one another, from OH, OR₁₁, NHR₁₃, NR₁₃R₁₄, C₁-C₈ alkyl, C₂-C₈ alkenyl, C₂-C₈, alkynyl, C₃-C₁₀ cycloalkyl, C₅-C₁₂ aryl, (C₅-C₁₂)-aryl-(C₁-C₈)-alkyl, (C₁-C₈)-alkyl-(C₅-C₁₂)-aryl, (C₁-C₈)-heteroalkyl, (C₃-C₈)-heterocycloalkyl, (C₅-C₁₂)-heteroaryl and NHCR_{α}R_{α′}—C(O)R₁₂; wherein:
   R₁₁ is selected from C₁-C₁₀ alkyl, C₃-C₁₀ cycloalkyl, C₅-C₁₂ aryl, (C₁-C₁₀)-alkyl-(C₅-C₁₂)-aryl, C₅-C₁₂ substituted aryl, C₁-C₁₀ heteroalkyl, C₁-C₁₀ haloalkyl, —(CH₂)ₘC(O)(C₁-C₁₅)-alkyl, —(CH₂)ₘOC(O)(C₁- C₁₅)-alkyl, —(CH₂)ₘOC(O)O(C₁-C₁₅)-alkyl, —(CH₂)ₘSC(O)(C₁-C₁₅)-alkyl, —(CH₂)ₘC(O)O(C₁-C₁₅)-alkyl, —(CH₂)ₘC(O)O(C₁-C₁₅)-alkyl-aryl; wherein m is an integer selected from 1 to 8; and P(O)(OH)OP(O)(OH)₂; an internal or external counter-ion;
   R₁₂ is selected from hydrogen, C₁-C₁₀ alkyl, C₂-C₈ alkenyl, C₂-C₈, alkynyl, C₁-C₁₀ haloalkyl, C₃-C₁₀ cycloalkyl, C₃-C₁₀ heterocycloalkyl, C₅-C₁₂ aryl, (C₁-C₄)-alkyl-(C₅-C₁₂)-aryl and C₅-C₁₂ heteroaryl; wherein said aryl or heteroaryl groups are optionally substituted by one or two groups selected from halogen, trifluoromethyl, C₁-C₆ alkyl, C₁-C₆ alkoxy and cyano;
   R₁₃ and R₁₄ are selected independently from H, C₁-C₈ alkyl and (C₁-C₈)-alkyl-(C₅-C₁₂)-aryl;
   R_{α} and R_{α′} selected independently, from hydrogen, C₁-C₁₀ alkyl, C₂-C₁₀ alkenyl, C₂-C₁₀ alkynyl, C₃-C₁₀ cycloalkyl, C₁-C₁₀ thio-alkyl, C₁-C₁₀ hydroxylalkyl, (C₁-C₁₀)-alkyl-(C₅-C₁₂)-aryl, C₅-C₁₂ aryl, —(CH₂)₃NHC(═NH)NH₂, (1H-indol-3-yl)-methyl, (1H-imidazol-4-yl)-methyl and a side chain selected from a proteinogenic or non-proteinogenic amino acid; wherein said aryl groups are optionally substituted by a group selected from hydroxyl, C₁-C₁₀ alkyl, C₁-C₆ alkoxy, halogen, nitro and cyano;
or R₉ and R₁₀, with the phosphorus atoms to which they are bonded, form a 6-member-ring, wherein —R₉-R₁₀— represents —CH₂—CH₂—CHR— or —O—CH₂—CH₂—CHR—O—; wherein R is selected from hydrogen, C₅-C₆ aryl and C₅-C₆ heteroaryl; wherein said aryl or heteroaryl groups are optionally substituted by one or two groups selected from halogen, trifluoromethyl, C₁-C₆ alkyl, C₁-C₆ alkoxy and cyano;
X′ is selected from O, CH₂, S, Se, CHF, CF₂ and C═CH₂;
R₁ is selected from H, azido, cyano, C₁-C₈ alkyl, C₁-C₈ thio-alkyl, C₁-C₈ heteroalkyl and OR; wherein R is selected from H and C₁-C₈ alkyl;
R_{2′}, R_{3′}, R_{4′} and R_{5′} are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein R_{AA} is a side chain selected from a proteinogenic amino acid;
R_{6′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{8′} is selected from H, OR, NHR₁₅—, NR₁₅—R₁₆—, NH—NHR₁₅—, SH, CN, N₃ and halogen; wherein R_{15′} and R_{16′} are selected, independently of one another, from H, C₁-C₈ alkyl and C₁-C₈ alkyl-aryl;
Y′ is selected from CH, CH₂, C(CH₃)₂ and CCH₃;
n is an integer selected from 1 to 3;
[symbol] represents a single or a double bond according to Y′; and
[symbol] represents the alpha or beta anomer according to the position of R_{1′};
R₈ is selected from H, OR, NHR₁₅, NR₁₅R₁₆, NH—NHR₁₅, SH, CN, N₃ and halogen; wherein R is selected from H and C₁-C₈ alkyl, and: R₁₅ and R₁₆ are selected, independently of one another, from H, C₁-C₈ alkyl and C₁-C₈ alkyl-aryl and —CHR_{AA}CO₂H wherein R_{AA} is a side chain selected from a proteinogenic or non-proteinogenic amino acid;
Y is selected from CH, CH₂, C(CH₃)₂ and CCH₃;
[symbol] represents a single or a double bond according to Y; and
[symbol] presents the alpha or beta anomer according to the position of R₁,
   for use thereof in the treatment of arrhythmia.

According to an embodiment, in the formula (I):
X is selected from O, CH₂, S, Se, CHF, CF₂ and C═CH₂;
R₁ is selected from H, azido, cyano, C₁-C₈ alkyl, C₁-C₈ thio-alkyl, C₁-C₈ heteroalkyl and OR; wherein R is selected from H and C₁-C₈ alkyl;
R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl aryl, C(O)NH(C₁-C₁₂)-alkyl aryl, C(O)O(C₁-C₁₂)-alkyl aryl and C(O)CHR_{A}NH₂; wherein RA is a side-chain selected from a proteinogenic amino acid;
R₆ is selected from H, azido, cyano, C₁-C₈ alkyl, C₁-C₈ thio-alkyl, C₁-C₈ heteroalkyl and OR; wherein R is selected from H and C₁-C₈ alkyl;
R₇ is selected from H, P(O)R₉R₁₀ and P(S)R₉R₁₀; wherein R₉ and R₁₀ are selected, independently of one another, from OH, OR₁₁, C₁-C₈ alkyl, C₅-C₁₂ aryl and NHCHR_{AA}C(O)R₁₂; wherein:
   R₁₁ is selected from C₁-C₈ alkyl, C₅-C₁₂ aryl and P(O)(OH)OP(O)(OH)₂;
   R₁₂ is a C₁-C₈ alkyl; and
   R_{AA} is a side chain selected from a proteinogenic amino acid;
R₈ is selected from H, OR, NHR_{13′}, NR_{13′}R_{14′}, NH—NHR_{13′}, SH, CN, N₃ and halogen; wherein R_{13′} and R_{14′} are selected, independently of one another, from H, C₁-C₈ alkyl and C₁-C₈ alkyl-aryl;
Y is selected from CH, CH₂, C(CH₃)₂ and CCH₃;
[symbol] represents a single or a double bond according to Y; and
[symbol] represents the alpha or beta anomer according to the position of R₁.

According to an embodiment, X is selected from O, CH₂ and S. In a preferred embodiment, X is oxygen.

According to an embodiment, R₁ and R₆ each represent, independently of one another, hydrogen or OH. In an embodiment, R₁ and R₆ each represent hydrogen.

According to an embodiment, R₁ is selected from hydrogen or OH. In an embodiment, R₁ is OH. In an embodiment, R₁ is hydrogen.

According to an embodiment, R₂, R₃, R₄ and R₅ are selected, independently of one another, from H, halogen, hydroxyl, C₁-C₁₂ alkyl and OR; wherein R is as defined above. In a preferred embodiment, R₂, R₃, R₄ and R₅ are selected, independently of one another, from H, hydroxyl and OR; wherein R is as defined above. In a more preferred embodiment, R₂, R₃, R₄ and R₅ are selected, independently of one another, from hydrogen or OH.

According to an embodiment, R₂ and R₃ are identical. In an embodiment, R₂ and R₃ are identical and represent OH. In an embodiment, R₂ and R₃ are identical and represent hydrogen.

According to an embodiment, R₂ and R₃ are different. In a preferred embodiment, R₂ is hydrogen and R₃ is OH. In a more preferred embodiment, R₂ is OH and R₃ is hydrogen.

According to an embodiment, R₄ and R₅ are identical. In an embodiment, R₄ and R₅ are identical and represent OH. In an embodiment, R₄ and R₅ are identical and represent hydrogen.

According to an embodiment, R₄ and R₅ are different. In a preferred embodiment, R₄ is OH and R₅ is hydrogen. In a more preferred embodiment, R₄ is hydrogen and R₅ is OH.

According to an embodiment, R₃ and R₄ are different. In an embodiment, R₃ is OH and R₄ is hydrogen. In an embodiment, R₃ is hydrogen and R₄ is OH.

According to an embodiment, R₃ and R₄ are identical. In a preferred embodiment, R₃ and R₄ are identical and represent OH. In a more preferred embodiment, R₃ and R₄ are identical and represent hydrogen.

According to an embodiment, R₂ and R₅ are different. In an embodiment, R₂ is hydrogen and R₅ is OH. In an embodiment, R₂ is OH and R₅ is hydrogen.

According to an embodiment, R₂ and R₅ are identical. In a preferred embodiment, R₂ and R₅ are identical and represent hydrogen. In a more preferred embodiment, R₂ and R₅ are identical and represent OH.

According to an embodiment, R₆ is selected from hydrogen or OH. In an embodiment, R₆ is OH. In a preferred embodiment, R₆ is hydrogen.

According to an embodiment, R₇ is selected from hydrogen, P(O)R₉R₁₀ and

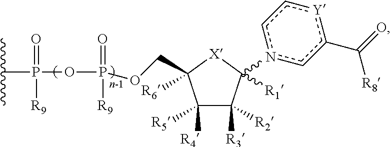

wherein R₉, R₁₀, R_{1′}-R_{6′}, R_{8′}, X′, Y′, n, [symbol] and [symbol] are as described above.

According to an embodiment, R₇ is selected from hydrogen or P(O)R₉R₁₀; wherein R₉ and R₁₀ are as defined above. In an embodiment, R₇ is selected from hydrogen or P(O)(OH)₂.

In an embodiment, R₇ is hydrogen. In another embodiment, R₇ is not hydrogen.

In an embodiment, R₇ is P(O)R₉R₁₀; wherein R₉ and R₁₀ are as defined above. In a preferred embodiment, R₇ is P(O)(OH)₂.

In another embodiment, R₇ is

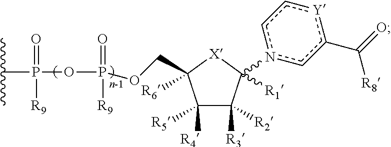

wherein R_{1′}, R_{2′}, R_{3′}, R_{4′}, R_{5′}, R_{6′}, R_{8′}, R₉, X′, Y′, n, [symbol] and [symbol] are as defined above.

In a particular embodiment, R₇ is

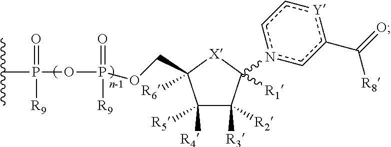

wherein:
X′ is selected from O, CH₂ and S; preferably X′ is oxygen;
R_{1′} is selected from H and OH, preferably R_{1′} is H;
R_{2′}, R_{3′}, R_{4′} and R_{5′} are selected, independently of one another, from H, halogen, hydroxyl, C₁-C₁₂ alkyl and OR; wherein R is as defined above; preferably R_{2′}, R_{3′}, R_{4′} and R_{5′} are selected, independently of one another, from H and OH;
R_{6′} is selected from H or OH; preferably R_{6′} is H;
R_{8′} is selected from H, OR, NHR_{15′} or NR_{15′}R_{16′}, wherein R_{15′} and R_{16′} are as described above; preferably R_{8′} is NH₂;
Y′ is selected from CH or CH₂;
n is an integer selected from 1 to 3; preferably n is equal to 2;
[symbol] represents a single or a double bond according to Y′; and
[symbol] represents the alpha or beta anomer according to the position of R_{1′};

According to an embodiment, n is equal to 1. According to an embodiment, n is equal to 2. According to an embodiment, n is equal to 3.

In an embodiment, Ra is selected from H, OR, NHR₁₅ and NR₁₅R₁₆; wherein R₁₅ and R₁₆ are as defined above. In a preferred embodiment, R₈ is NHR₁₅; wherein R₁₅ is as defined above. In a preferred embodiment, R₈ is NH₂.

In an embodiment, Y is CH. In an embodiment, Y is CH₂.

According to a preferred embodiment, in the formulas of the present application and, in particular, the formulas detailed below, R₇ is not hydrogen.

In a preferred embodiment, the compounds of formula (I) are compounds of formula (I-1):

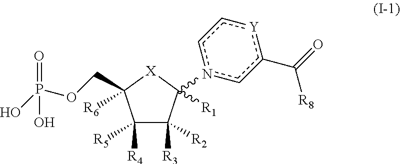

or a pharmaceutically acceptable salt and/or solvate thereof, wherein R₁, R₂, R₃, R₄, R₅, R₆, R₈, X, Y, [symbol] and [symbol] are as defined above for the compounds of formula (I).

In a preferred embodiment, the compounds of formula (I) are compounds of formula (I-2):

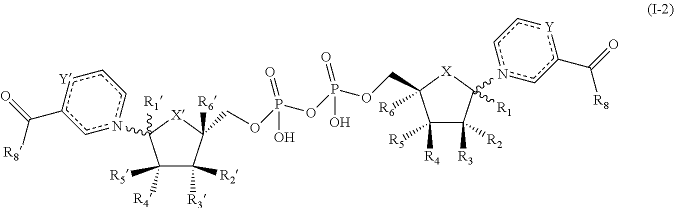

or a pharmaceutically acceptable salt and/or solvate thereof, wherein R₁, R₂, R₃, R₄, R₅, R₆, R₈, X, Y, R_{1′}, R_{2′}, R_{3′}, R_{4′}, R_{5′}, R_{6′}, R_{8′}, X′, Y′, [symbol] and [symbol] are as define above for the compounds of formula (I).

In a preferred embodiment, the compounds of formula (I) are those in which X represents oxygen.

In a preferred embodiment, among the compounds of formula (I), the invention also relates to a compound of formula (II):

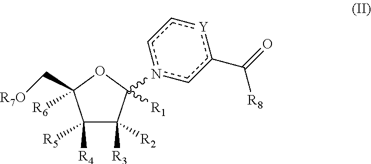

or a pharmaceutically acceptable salt and/or solvate thereof, wherein R₁, R₂, R₃, R₄, R₅, R₆, R₇, R₈, Y, [symbol] and [symbol] are as defined above for the compounds of formula (I).

In a preferred embodiment, the compounds of formula (II) are compounds of formula (II-1):

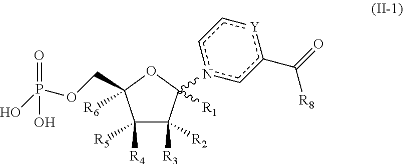

or a pharmaceutically acceptable salt and/or solvate thereof, wherein R₁, R₂, R₃, R₄, R₅, R₆, R₈, Y, [symbol] and [symbol] are as defined above for the compounds of formula (I).

In a preferred embodiment, the compounds of formula (II) are compounds of formula (II-2):

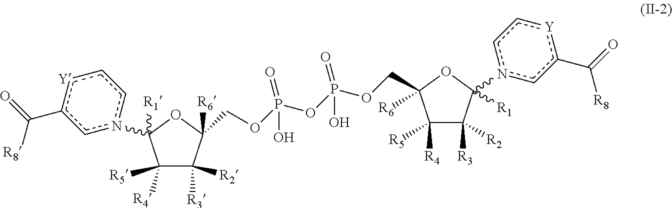

or a pharmaceutically acceptable salt and/or solvate thereof, wherein R₁, R₂, R₃, R₄, R₅, R₆, R₈, Y, R_{1′}, R_{2′}, R_{3′}, R_{4′}, R_{5′}, R_{6′}, R_{8′}, Y′, [symbol] and [symbol] are as defined above for the compounds of formula (I).

In a preferred embodiment, the compounds of formula I are those in which R₁ is hydrogen.

In a preferred embodiment, among the compounds of formula (I), the invention also relates to a compound of formula (III):

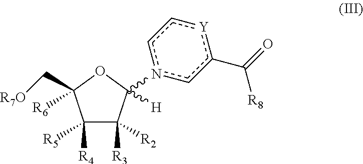

or a pharmaceutically acceptable salt and/or solvate thereof, wherein R₂, R₃, R₄, R₅, R₆, R₇, R₈, Y, [symbol] and [symbol] are as defined above for the compounds of formula (I).

In a preferred embodiment, the compounds of formula (III) are compounds of formula (III-1):

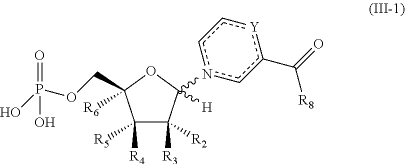

or a pharmaceutically acceptable salt and/or solvate thereof, wherein R₂, R₃, R₄, R₅, R₆, R₈, Y, [symbol] and [symbol] are as defined above for the compounds of formula (I).

In a preferred embodiment, the compounds of formula (III) are compounds of formula (III-2):

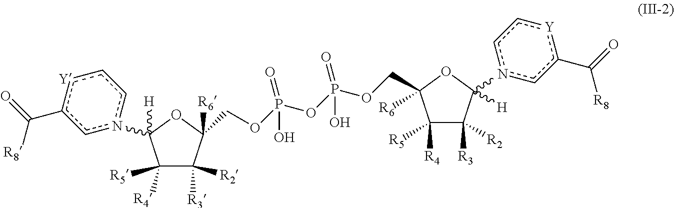

or a pharmaceutically acceptable salt and/or solvate thereof, wherein R₂, R₃, R₄, R₅, R₆, R₈, Y, R_{2′}, R_{3′}, R_{4′}, R_{5′}, R_{6′}, R_{8′}, Y′, [symbol] and [symbol] are as defined above for the compounds of formula (I).

In a preferred embodiment, the compounds of formula (I) are those in which R₂ is OH and R₃ is hydrogen.

In a preferred embodiment, the compounds of formula (I) are those in which R₄ is hydrogen and R₅ is OH.

In a preferred embodiment, the compounds of formula (I) are those in which R₃ and R₄ are identical and represent hydrogen.

In a preferred embodiment, among the compounds of formula (I), the invention also relates to a compound of formula (IV):

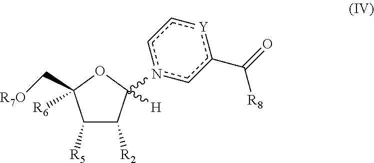

or a pharmaceutically acceptable salt and/or solvate thereof, wherein R₂, R₅, R₆, R₇, R₈, Y, [symbol] and [symbol] are as defined above for the compounds of formula (I).

In a preferred embodiment, the compounds of formula (IV) are compounds of formula (IV-1):

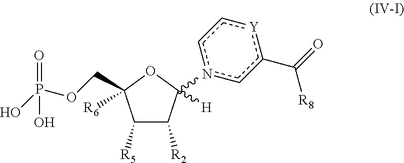

or a pharmaceutically acceptable salt and/or solvate thereof, wherein R₂, R₅, R₆, R₈, Y, [symbol] and [symbol] are as defined above for the compounds of formula (I).

In a preferred embodiment, the compounds of formula (IV) are compounds of formula (IV-2):

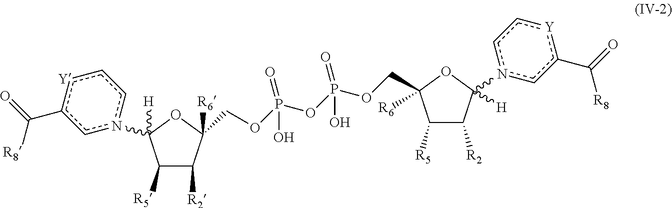

or a pharmaceutically acceptable salt and/or solvate thereof, wherein R₂, R₅, R₆, R₈, Y, R_{2′}, R_{5′}, R_{6′}, R_{8′}, Y′, [symbol] and [symbol] are as defined above for the compounds of formula (I).

In a preferred embodiment, the compounds of formula (I) are those in which R₂ and R₅ are identical and represent OH.

In a preferred embodiment, among the compounds of formula (I), the invention also relates to a compound of formula (V):

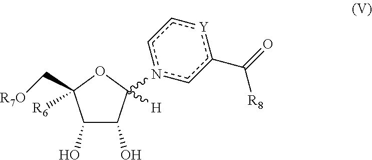

or a pharmaceutically acceptable salt and/or solvate thereof, wherein R₆, R₇, R₈, Y, [symbol] and [symbol] are as defined above for the compounds of formula (I).

In a preferred embodiment, the compounds of formula (V) are compounds of formula (V-1):

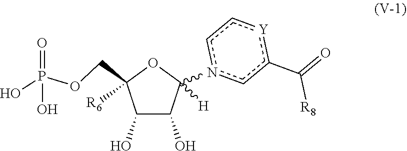

or a pharmaceutically acceptable salt and/or solvate thereof, wherein R₆, R₇, R₈, Y, [symbol] and [symbol] are as defined above for the compounds of formula (I).

In a preferred embodiment, the compounds of formula (V) are compounds of formula (V-1):

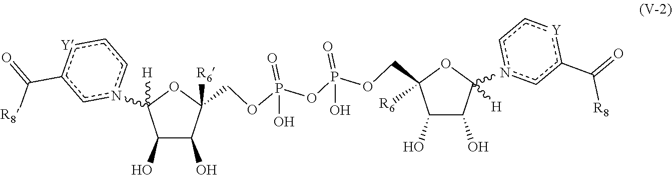

or a pharmaceutically acceptable salt and/or solvate thereof, wherein R₆, R₈, Y, R_{6′}, R_{8′}, Y′, [symbol] and [symbol] are as defined above for the compounds of formula (I).

In a preferred embodiment, the compounds of formula (I) are those in which R₆ is hydrogen.

In a preferred embodiment, among the compounds of formula (I), the invention also relates to a compound of formula (VI):

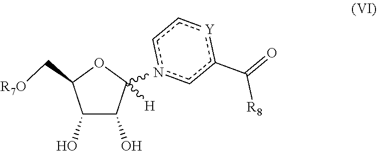

or a pharmaceutically acceptable salt and/or solvate thereof, wherein R₇, R₈, Y, [symbol] and [symbol] are as defined above for the compounds of formula (I).

In a preferred embodiment, the compounds of formula (VI) are compounds of formula (VI-1):

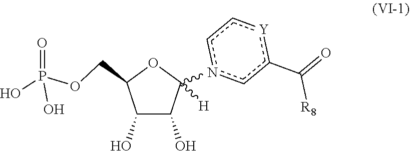

or a pharmaceutically acceptable salt and/or solvate thereof, wherein R₈, Y, [symbol] and [symbol] are as defined above for the compounds of formula (I).

In a preferred embodiment, the compounds of formula (VI) are compounds of formula (VI-2):

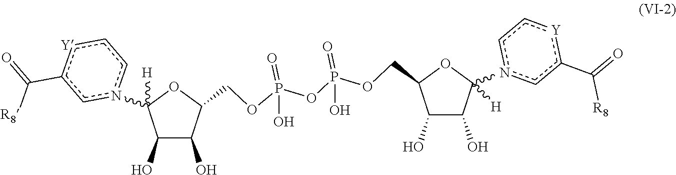

or a pharmaceutically acceptable salt and/or solvate thereof, wherein R₈, Y, [symbol] an [symbol] are as defined above for the compounds of formula (I).

In a preferred embodiment, the compounds of formula (I) are those in which R₈ is NH₂.

In a preferred embodiment, among the compounds of formula (I), the invention also relates to a compound of formula (VII):

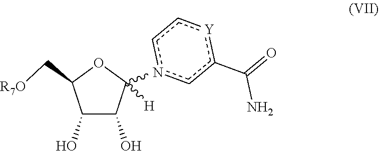

or a pharmaceutically acceptable salt and/or solvate thereof, wherein R₇, Y, [symbol] and [symbol] are as defined above for the compounds of formula (I).

In a preferred embodiment, the compounds of formula (VII) are compounds of formula (VII-1):

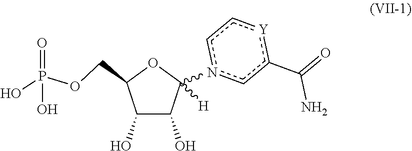

or a pharmaceutically acceptable salt and/or solvate thereof, wherein Y, [symbol] and [symbol] are as defined above for the compounds of formula (I).

In a preferred embodiment, the compounds of formula (VII) are compounds of formula (VII-2):

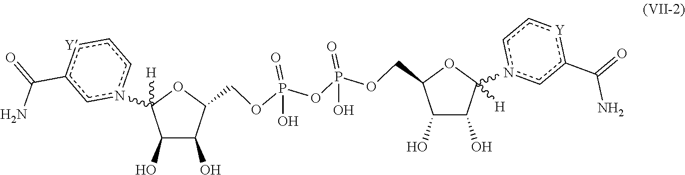

or a pharmaceutically acceptable salt and/or solvate thereof, wherein Y, Y′, [symbol] and [symbol] are as defined above for the compounds of formula (I).

In a preferred embodiment, the compounds of formula (I) are those in which Y is CH.

In a preferred embodiment, among the compounds of formula (I), the invention also relates to a compound of formula (VIII):

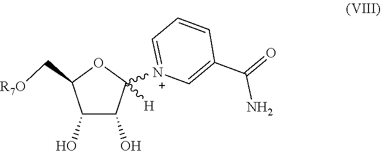

or a pharmaceutically acceptable salt and/or solvate thereof, wherein R₇ and [symbol] are as defined above for the compounds of formula (I).

In a preferred embodiment, the compounds of formula (VIII) are compounds of formula (VIII-1):

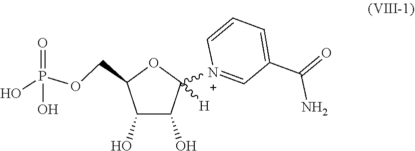

or a pharmaceutically acceptable salt and/or solvate thereof, wherein [symbol] is as defined above for the compounds of formula (I).

In a preferred embodiment, the compounds of formula (VIII) are compounds of formula (VIII-2):

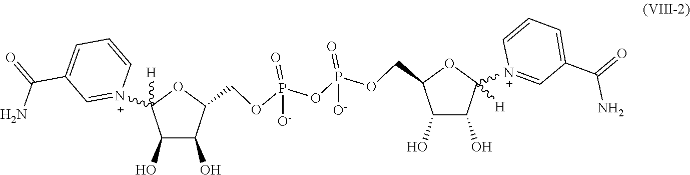

or a pharmaceutically acceptable salt and/or solvate thereof, wherein [symbol] is as defined above for the compounds of formula (I).

In a preferred embodiment, the compounds of formula (I) are those in which Y is CH₂.

In a preferred embodiment, among the compounds of formula (I), the invention also relates to a compound of formula (IX):

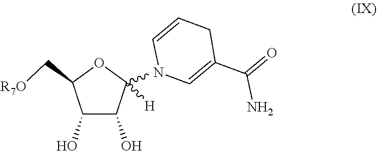

or a pharmaceutically acceptable salt and/or solvate thereof, wherein R₇ and [symbol] are as defined above for the compounds of formula (I).

In a preferred embodiment, the compounds of formula (IX) are compounds of formula (IX-1):

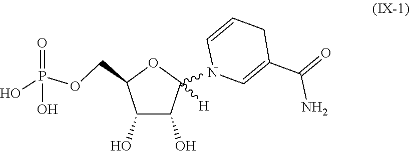

or a pharmaceutically acceptable salt and/or solvate thereof, wherein [symbol] is as defined above for the compounds of formula (I).

In a preferred embodiment, the compounds of formula (IX) are compounds of formula (IX-2):

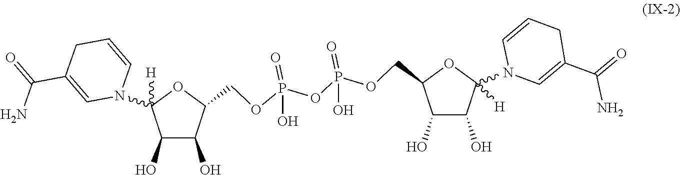

or a pharmaceutically acceptable salt and/or solvate thereof, wherein [symbol] is as defined above for the compounds of formula (I).

In a particular embodiment, the compounds of formula (I) are those in which R₇ is hydrogen.

In a preferred embodiment, among the compounds of formula (I), the invention also relates to a compound of formula (X):

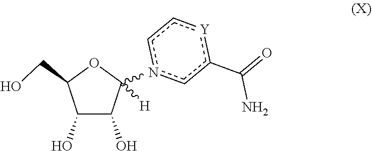

or a pharmaceutically acceptable salt and/or solvate thereof, wherein Y, [symbol] and [symbol] are as defined above for the compounds of formula (I).

According to an embodiment, the compounds of the invention are selected from the compounds of Table 2 below or a pharmaceutically acceptable salt and/or solvate thereof:

TABLE 2

| Compounds | |
|---|---|
| (anomers) | Structure |
| I-A (beta) | 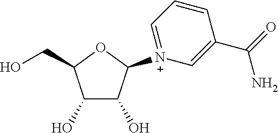 [SMILES: NC(=O)c1ccc[n+]([C@@H]2O[C@H](CO)[C@@H](O)[C@H]2O)c1] |
| I-B (alpha) | 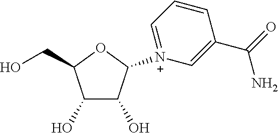 [SMILES: NC(=O)c1ccc[n+]([C@H]2O[C@H](CO)[C@@H](O)[C@H]2O)c1] |
| I-C (beta) | 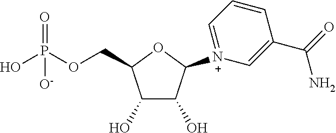 [SMILES: NC(=O)c1ccc[n+]([C@@H]2O[C@H](COP(=O)([O-])O)[C@@H](O)[C@H]2O)c1] |
| I-D (alpha) | 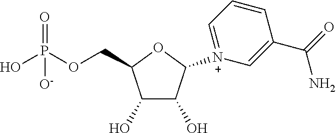 [SMILES: NC(=O)c1ccc[n+]([C@H]2O[C@H](COP(=O)([O-])O)[C@@H](O)[C@H]2O)c1] |
| I-E (beta) | 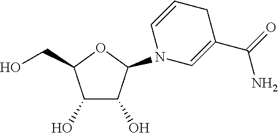 [SMILES: NC(=O)C1=CN([C@@H]2O[C@H](CO)[C@@H](O)[C@H]2O)C=CC1] |
| I-F (alpha) | 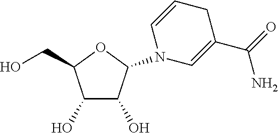 [SMILES: NC(=O)C1=CN([C@H]2O[C@H](CO)[C@@H](O)[C@H]2O)C=CC1] |
| I-G (beta) | 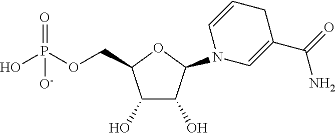 [SMILES: NC(=O)C1=CN([C@@H]2O[C@H](COP(=O)([O-])O)[C@@H](O)[C@H]2O)C=CC1] |
| I-H (alpha) | 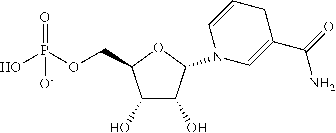 [SMILES: NC(=O)C1=CN([C@H]2O[C@H](COP(=O)([O-])O)[C@@H](O)[C@H]2O)C=CC1] |
| I-I (beta, beta) | 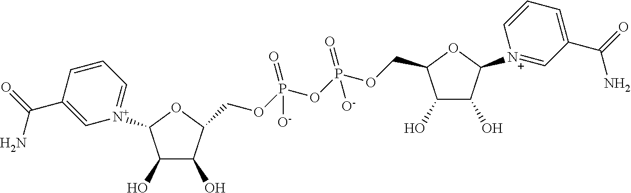 [SMILES: NC(=O)c1ccc[n+]([C@@H]2O[C@H](COP(=O)([O-])OP(=O)([O-])OC[C@H]3O[C@@H]([n+]4cccc(C(N)=O)c4)[C@H](O)[C@@H]3O)[C@@H](O)[C@H]2O)c1] |
| I-J (beta, alpha) | 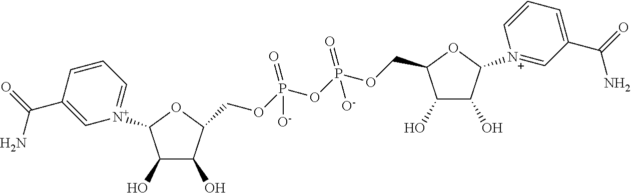 [SMILES: NC(=O)c1ccc[n+]([C@@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] |
| I-K (alpha, alpha) | 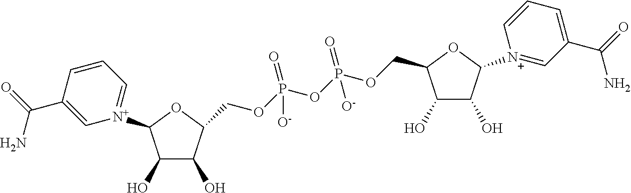 [SMILES: NC(=O)c1ccc[n+]([C@H]2O[C@H](COP(=O)([O-])OP(=O)([O-])OC[C@H]3O[C@H]([n+]4cccc(C(N)=O)c4)[C@H](O)[C@@H]3O)[C@@H](O)[C@H]2O)c1] |
| I-L (beta, beta) | 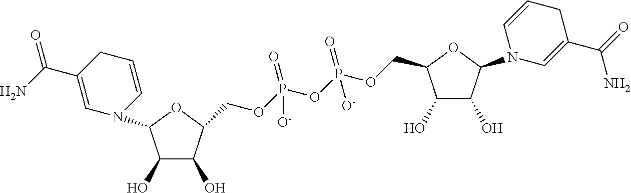 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| I-M (beta, alpha) | 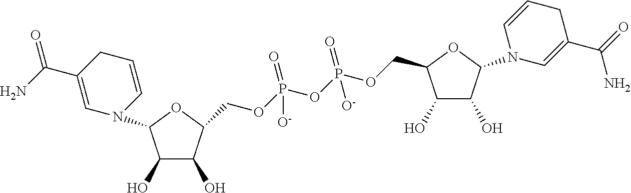 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| I-N (alpha, alpha) | 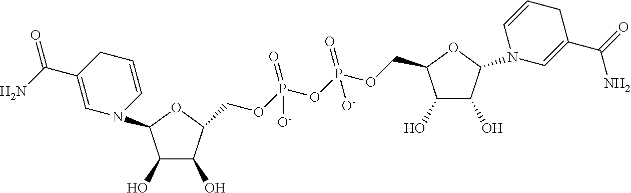 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |

In a preferred embodiment, the compounds of the invention are the compounds of formula I-A, I-C, I-E and I-G of Table 2 above or a pharmaceutically acceptable salt and/or solvate thereof.

In a preferred embodiment, the compounds of the invention are the compounds of formula I-C and I-G of Table 2 above or a pharmaceutically acceptable salt and/or solvate thereof.

In a preferred embodiment, the compounds of the invention are the compounds of formula I-C and I-D of Table 2 above or a pharmaceutically acceptable salt and/or solvate thereof.

In a more preferred embodiment, the compound of the invention is the compound of formula I-C or a pharmaceutically acceptable salt and/or solvate thereof.

In a preferred embodiment, the compounds of the invention are the compounds of formula I-I, I-J, I-K, I-L, I-M and I-N of Table 2 above or a pharmaceutically acceptable salt and/or solvate thereof. In a preferred embodiment, the compounds of the invention are the compounds of formula I-I, I-J and I-K of Table 2 above or a pharmaceutically acceptable salt and/or solvate thereof.

In a preferred embodiment, the compounds of the invention are the compounds of formula I-C, I-D, I-I, I-J and I-K of Table 2 above or a pharmaceutically acceptable salt and/or solvate thereof.

Pharmaceutical Composition for Treating Arrhythmia

According to another embodiment, the present invention relates to a pharmaceutical composition comprising at least one compound of the invention and at least one pharmaceutically acceptable excipient.

According to another embodiment, the present invention relates to a drug comprising at least one compound of the invention.

In an embodiment, the pharmaceutical composition of the invention or the drug of the invention comprises, in addition, at least one compound of the invention as active substances, therapeutic agents and/or additional active substances. Non-limiting examples of therapeutic agents and/or additional active substances comprise selective inhibitors of Na⁺/H⁺ exchange, such as cariporide, zoniporide or amiloride; beta-blockers, such as atenolol, metoprolol; calcium inhibitors such as verapamil or anticoagulants.

Method

According to another aspect, the invention relates to a method for preparing compounds of formula (I) as described above.

In particular, the compounds of formula (I) disclosed herein can be prepared as described below from substrates A-E. A person skilled in the art would understand that these reaction schemes are in no way limiting and the variations can be made without departing from the spirit and scope of the present invention.

According to an embodiment, the invention relates to a method for preparing compounds of formula (I) as described above.

The method involves, in a first step, the mono-phosphorylation of a compound of formula (A), in the presence of phosphoryl chloride and trialkyl phosphate, to lead to the phosphorodichloridate of formula (B),

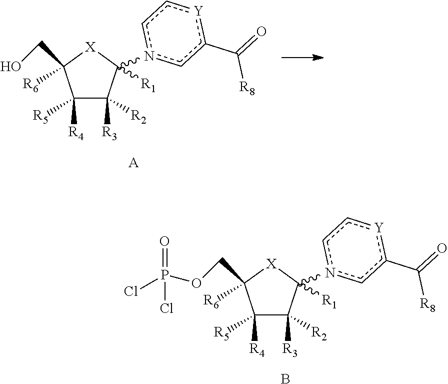

wherein X, R₁, R₂, R₃, R₄, R₅, R₆, R₈, Y, [symbol] and [symbol] are as defined above for the compounds of formula (I).

In a second step, the phosphorodichloridate of formula (B) is hydrolysed to lead to the phosphate of formula (C),

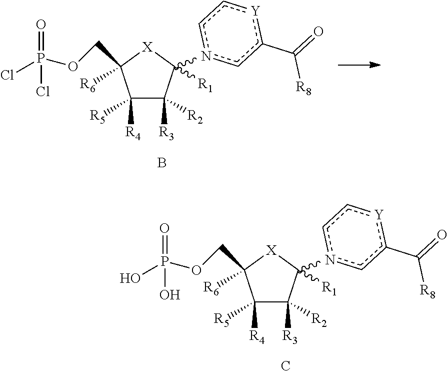

wherein X, R₁, R₂, R₃, R₄, R₆, R₈, Y, [symbol] and [symbol] are as defined above for the compounds of formula (I).

According to an embodiment, the compound of formula (A) is synthesised using various methods known to a person skilled in the art.

According to an embodiment, the compound of formula (A) is synthesised by reacting the pentose of formula (D) with a nitrogenous derivative of formula (E), wherein R, R₂, R₃, R₄, R₅, R₆, R₇, Y are as described above for the compounds of formula I, leading to the compound of formula (A-1) which is then selectively deprotected in order to give the compound of formula (A),

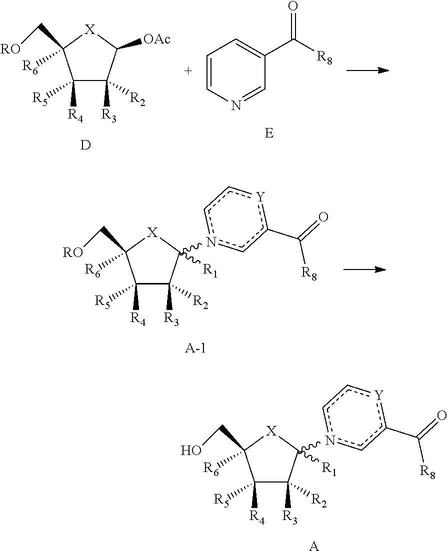

wherein X, R₁, R₂, R₃, R₄, R₅, R₆, R₈, Y, [symbol] and [symbol] are as defined above for the compounds of formula (I).

According to an embodiment, R is a suitable protective group known to a person skilled in the art. In an embodiment, the protective group is selected from triarylmethyls and/or silyls. Non-limiting examples of triarylmethyl include the trityl, monomethoxytrityl, 4,4′-dimethoxytrityl and 4,4′,4″-trimethoxytrityl groups. Non-limiting examples of silyl groups comprise the trimethylsilyl, tert-butyldimethylsilyl, triisopropylsilyl, tert-butyldiphenylsilyl, tri-iso-propylsilyloxymethyl and [2-(trimethylsilyl)ethoxy]methyl groups.

According to an embodiment, any hydroxyl group attached to the pentose is protected by a suitable protective group known to a person skilled in the art.

The choice and exchange of protective groups is within the skill of a person skilled in the art. The protective groups can also be removed by methods that are well-known to a person skilled in the art, for example with an acid (for example, a mineral or organic acid), a base or fluoride source.

In a preferred embodiment, the nitrogenous derivative of formula (E) is coupled to the pentose of formula (D) by a reaction in the presence of a Lewis acid leading to the compound of formula (A-1). Non-limiting examples of Lewis acids include TMSOTf, BF₃.OEt₂, TiCl₄ and FeCl₃.

In an embodiment, the method of the present invention further comprises a step of reducing the compound of formula (A) by various methods that are well known to a person skilled in the art, leading to the compound of formula (A′) which contains CH₂, and R₁, R₂, R₃, R₄, R₅, R₆, R₈, Y, [symbol] and [symbol] are as defined above for the compounds of formula (I).

In a particular embodiment, the present invention relates to a method for preparing compounds of formula I-A, I-C, I-E, I-G.

In a first step, the nicotinamide of formula E is coupled to the ribose tetraacetate of formula D by a coupling reaction in the presence of a Lewis acid, leading to the compound of formula A-1:

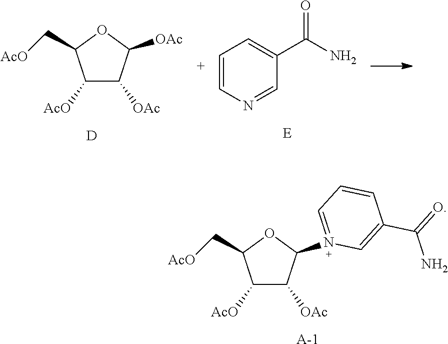

In a second step, an ammonia treatment of the compound of formula A-1 is carried out, leading to the compound of formula I-A:

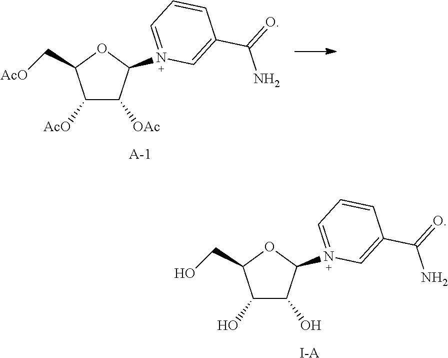

In a third step, the mono-phosphorylation of the compound of formula I-A, in the presence of phosphoryl chloride and a trialkyl phosphate, leads to the phosphorodichloridate of formula I-A′:

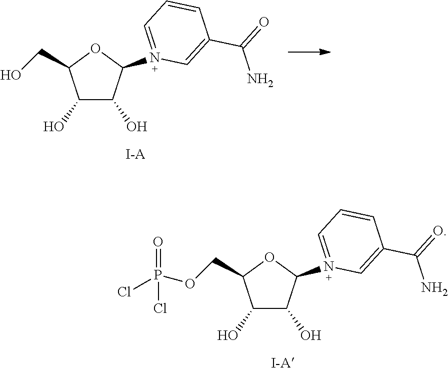

In a fourth step, the phosphorodichloridate of formula B is hydrolysed in order to give the compound of formula I-C:

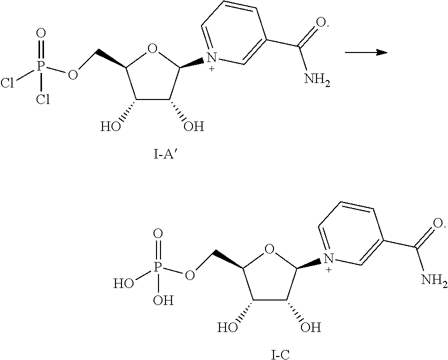

In an embodiment, a step of reducing the compound of formula I-A is carried out, leading to the compound of formula I-E.

The compound of formula I-E is then monophosphorylated as described for the fourth step and hydrolysed in order to give the compound of formula I-G.

According to an embodiment, the compounds of the invention can be prepared by any suitable method, in particular those described in applications EP 19218817.5, EP 20190010.7 and EP 20215832.5.

Use

The present invention thus relates to the compounds of the invention for use thereof in the treatment of arrhythmia.

According to an embodiment, the present invention relates to compounds of formula (I)-(XI) or a pharmaceutically acceptable salt and/or solvate thereof, as described above, for use thereof in the treatment of arrhythmia.

In an embodiment, the present invention relates to compounds of formula (I)-(XI) or a pharmaceutically acceptable salt and/or solvate thereof, as described above, for use thereof in the prophylactic treatment of arrhythmia.

In an embodiment, the present invention relates to compounds of formula (I)-(XI) or a pharmaceutically acceptable salt and/or solvate thereof, as described above, for use thereof in the treatment of the ventricular tachycardia.

In an embodiment, the present invention relates to compounds of formula (I)-(XI) or a pharmaceutically acceptable salt and/or solvate thereof, as described above, for use thereof in the prophylactic treatment of the ventricular tachycardia.

In an embodiment, the present invention relates to compounds of formula (I)-(XI) or a pharmaceutically acceptable salt and/or solvate thereof, as described above, for use thereof in the treatment of the ventricular fibrillation.

In an embodiment, the present invention relates to compounds of formula (I)-(XI) or a pharmaceutically acceptable salt and/or solvate thereof, as described above, for use thereof in the prophylactic treatment of the ventricular fibrillation.

According to another embodiment, the present invention relates to a pharmaceutical composition comprising at least one compound of the invention, and at least one pharmaceutically acceptable excipient for use thereof in the treatment of arrhythmia.

According to another embodiment, the present invention relates to a drug comprising at least one compound of the invention for use thereof in the treatment of arrhythmia.

In an embodiment, the pharmaceutical composition of the invention or the drug of the invention comprises, in addition, at least one compound of the invention as active substances, therapeutic agents and/or additional active substances. Non-limiting examples of therapeutic agents and/or additional active substances include the selective inhibitors of Na⁺/H⁺ exchange, such as cariporide, zoniporide and amiloride.

According to an embodiment, the present invention relates to the use of the compounds of the invention as described above for the treatment of arrhythmia. In an embodiment, the present invention relates to the use of the compounds of the invention as described above for the prophylactic treatment de arrhythmia.

According to another embodiment, the present invention relates to the use of a pharmaceutical composition comprising at least one compound of the invention and at least one pharmaceutically acceptable excipient for the treatment of arrhythmia.

According to another embodiment, the present invention relates to the use of a drug comprising at least one compound of the invention for the treatment of arrhythmia.

In an embodiment, the present invention relates to the use of the compounds of the invention as described above for the manufacture of a drug for the treatment of arrhythmia.

The present invention also relates to a method for treating arrhythmia in a subject in need thereof, said method comprising administering to said subject a therapeutically effective quantity of at least one compound or a composition of the invention as described above.

In an embodiment, the subject who is in need of a therapeutic or preventive treatment is diagnosed by a health professional. In practice, arrhythmia is diagnosed by any examination routinely performed in the medical environment, in particular an electrocardiogram, stress test or electrophysiological examination.

Preferably, the subject is a warm-blooded animal, more preferably a human.

According to an embodiment, the compounds of the invention can be administered within the framework of a combined therapy in which one or more compounds of the invention or a composition or a drug which contains a compound of the present invention, as active substances, are co-administered in combination with therapeutic agents and/or additional active substances.

In an embodiment, the compounds of the invention, the pharmaceutical composition of the invention or the drug of the invention are used as a complement to the ablation of the abnormal tissue.

In an embodiment, the compounds of the invention are not administered within the framework of a combined therapy comprising the administration of plasma, NAD and/or NAD promoter. “NAD” refers to the co-enzyme nicotinamide adenine dinucleotide. An “NAD promoter” refers, in particular, to one or more of the following: a positive regulator of NAMPT, a negative regulator of NADase, a negative regulator of NNMT (nicotinamide N-methyltransferase), a positive regulator of NMN AT s 1-3 (nicotinamide mononucleotide adenylyltransferase), an inhibitor of Cx43 (connexin 43), a positive regulator of CD73, a negative regulator of CD 157, a positive regulator of the kinase protein activated by AMP 5′(AMPK), a positive regulator of NR kinase1/2 (NRK1/2), a positive regulator of NARPT, a positive regulator of quinolinate phosphoribosyl transferase (QPRT), a positive regulator of NAD synthase 1 (NADSyn1), a negative regulator of miARN-34a, a positive regulator of purine nucleoside phosphorylase (PNP) and a positive regulator of NQO1; and any combination thereof. The term “negative regulator” means an inhibitor or suppressor; and the term “positive regulator” means an activator or promoter.

In the above-described embodiments, the compound of the invention and other therapeutic active agents can be administered in terms of dosage forms, either separately or in association with one another and, in terms of administration times, either sequentially or simultaneously.

Generally, for a pharmaceutical use, the compounds of the invention can be formulated in the form of a pharmaceutical preparation comprising at least one compound of the invention and at least one pharmaceutically acceptable excipient and optionally one or more other pharmaceutically active compounds.

By way of non-limiting examples, such a formulation can be in a form suitable for oral administration, parenteral administration (for example by intravenous, intramuscular or subcutaneous injection or by intravenous perfusion), for topical administration (including ocular), for administration by inhalation, by means of a skin patch, via an implant, via a suppository, etc. These suitable forms of administration, which may be solid, semi-solid or liquid depending on the method of administration, as well as the methods and supports, diluents and excipients to be used for their preparation, will be clear to a person skilled in the art; reference is made to the latest edition of Remington's Pharmaceutical Sciences.

Preferred, but not limiting, examples of such preparations include, tablets, pills, powders, lozenges, sachets, wafer capsules, elixirs, suspensions, emulsions, solutions, syrups, ointments, creams, lotions, soft and hard gelatin capsules, sterile injectable solutions and sterile packaged powders (which are generally reconstituted before use) for bolus administration and/or for continuous administration, which can be formulated with supports, excipients and diluents which are suitable per se for such formulations, such as lactose, dextrose, sucrose, sorbitol, mannitol, starches, gum acacia, calcium phosphate, alginates, tragacanth gum, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, food oils, vegetable and mineral oils or the suitable mixtures thereof. The formulations can optionally contain other substances commonly used in pharmaceutical formulations, such as lubricants, wetting agents, emulsifiers and suspension agents, dispersants, disintegrating agents, bulking agents, filling agents, preservatives, sweeteners, flavourings, flow regulators, mould release agents, etc. The compositions can also be formulated so as to ensure a quick, prolonged or delayed release of the one or more active compounds that they contain.

The pharmaceutical preparations of the invention are preferably in the form of unitary doses and can be suitably packaged, for example in a box, blister, bottle, sachet, ampoule or any other suitable single-dose or multiple-dose support or receptacle (which can be correctly labelled); optionally with one or more leaflets containing information on the product and/or instructions for use. Generally, these unitary doses contained between 1 and 1000 mg, and generally between 1 and 500 mg, preferably between 250 and 500 mg of at least one compound of the invention.

In practice, the effective dose to be administered depends on one or more parameters including, in particular, the equipment used for the administration, age, sex, height, weight, physical condition and degree of severity of the disorder to be treated.

In general, the active compound of the invention will be administered between 0.1 mg per kilogram and 5000 mg per kilogram of body weight, more often between 1 mg per kilogram and 2000 mg per kilogram of body weight, preferably between 1 and 100 mg per kilogram of body weight, for example approximately 1, 10, 100 mg per kilogram of body weight of the human patient per day, which can be administered in a single daily dose, divided into one or more daily doses, or essentially continuously, for example by using a drip perfusion.

### BRIEF DESCRIPTION OF THE FIGURES

FIG. 1 is a histogram showing the incidence (FIG. 1A) and duration (FIG. 1B) of the ventricular tachycardia analysed during the ischaemia.

FIG. 2 is a histogram showing the incidence (FIG. 2A) and duration (FIG. 2B) of the ventricular fibrillation analysed during the ischaemia.

FIG. 3 is a histogram showing the incidence (FIG. 3A) and duration (FIG. 3B) of the ventricular tachycardia analysed during the reperfusion.

FIG. 4 is a histogram showing the incidences (FIG. 4A) and duration (FIG. 4B) of the ventricular fibrillation analysed during the reperfusion, as well as the number of ventricular fibrillations (FIG. 4C) during this period.

FIG. 5 is a histogram showing the mortality rate of rats treated with a saline solution, the compound of formula I-C and cariporide.

FIG. 6 is a histogram showing the heart rate 5 days after the injection of the carrier or DOX (20 mg/kg). *** p<0.001: t-test or Mann-Whitney test-DOX mice treated with the carrier vs control mice, $$$ p<0.001: Unidirectional ANOVA followed by a post-hoc Dunnett test or Kruskal-Wallis test followed by a post-hoc Dunn test-DOX treated mice with the carrier vs DOX mice treated with NMN analogues (180 mg/kg) or a carrier.

### EXAMPLES

The present invention will be better understood on reading the following examples which illustrate the invention in a non-limiting manner.

### I. Synthesis of the Compounds of the Invention

### 1. Material and Methods

All the chemicals were obtained from commercial suppliers and users without further purification. Thin layer chromatography was carried out on plastic sheets of TLC silica gel 60 F254 (layer thickness 0.2 mm) from Merck. Purification by column chromatography was carried out on the silica gel 60 (70-230 mesh ASTM, Merck). The melting points were determined either on a digital apparatus (Electrothermal IA 8103) and are not corrected, or on a WME Kofler bench (Wagner & Munz). The IR, ¹H, ¹⁹F and ¹³C NMR spectra confirmed the structure of all the compounds. The IR spectra were recorded on a Perkin Elmer Spectrum 100 FT-IR spectrometer and the NMR spectra were recorded, using CDCl₃, CD₃CN, D₂O or DMSO-d₆ as solvent, on a BRUKER AC 300 or 400 spectrometer at 300 or 400 MHz for the ¹H spectrum, 75 or 100 MHz for the ¹³C spectrum and 282 or 377 MHz for the ¹⁹F spectrum. The chemical shifts (δ) were expressed in parts per million with respect to the signal, indirectly (i) to CHCl₃ (δ 7.27) for ¹H and (ii) to CDCl₃ (δ 77.2) for ¹³C and directly (iii) to CFCl₃ (internal standard) (δ 0) for ¹⁹F. The chemical shifts are given in ppm and the multiplicities of peaks are designated as follows: s, singlet; br s, wide singlet; d, doublet; dd, doublet of doublet; t, triplet; q, quadruplet; quint, quintuplet; m, multiplet. High resolution mass spectra (HRMS) were obtained from the “Service central d'analyse de Solaize” (Centre national de la recherche scientifique) and have been recorded on a Waters spectrometer, using electrospray ionisation-TOF (ESI-TOF).

### General Procedure

### Step 1: Synthesis of the Compound of Formula A-1

The compound of formula D (1.0 equiv.) is dissolved in dichloromethane. The nicotinamide of formula E (1.50 equiv.) and TMSOTf (1.55 equiv.) are added at ambient temperature. The mixture is heated with reflux and stirred until the reaction is achieved. The mixture is cooled to ambient temperature and filtered. The filtrate is concentrated to dryness to give tetraacetate A-1.

### Step 2: Synthesis of the Compound of Formula I-A

The tetraacetate A-1 is dissolved in methanol and cooled to −10° C. 4.6 M ammonia in methanol (3.0 equivalents) at −10° C. is added and the mixture is stirred at this temperature until the reaction is complete. Dowex HCR (H+) resin is added to a pH of 6-7. The reaction mixture is heated to 0° C. and filtered. The resin is washed with a mixture of methanol and acetonitrile. The filtrate is concentrated to dryness. The residue is dissolved in acetonitrile and concentrated to dryness. The residue is dissolved in acetonitrile to give a solution of the compound of formula I-A.

### Step 3: Synthesis of the Compound of Formula I-A′

The crude solution of the compound of formula I-A in acetonitrile is diluted with trimethyl phosphate (10.0 equivalents). The acetonitrile is distilled under vacuum and the mixture is cooled to −10° C. Phosphorus oxychloride (4.0 equivalents) is added at −10° C. and the mixture is stirred at −10° C. until the reaction is ended.

### Step 4 and S: Synthesis of the Compound of Formula I-C

The mixture is hydrolysed by the addition of a 50/50 mixture of acetonitrile and water, followed by the addition of methyl tert-butyl ether. The mixture is filtered and the solid is dissolved in water. The aqueous solution is neutralised by the addition of sodium bicarbonate and extracted with dichloromethane. The aqueous layer is concentrated to dryness in order to give the crude compound of formula I-C, which is purified on a DOWEX 50w×8 column with elution in water followed by a silica gel chromatograph column.

### II. Evaluation of the Compounds of the Invention in a Model of Arrhythmia Induced by Ischaemia-Reperfusion

The aim of this study was to evaluate whether the administration of NAD precursor can attenuate the appearance of arrhythmia in an ischaemia-reperfusion rat.

All the procedures have been carried out in accordance with the Guidelines for the care and use of laboratory animals (revised in 1996 and 2011, 2010/63/UE) and with French law.

### 1. Material and Methods

### 1.1. Materials

### 1.1.1. Products Tested

TABLE 3

| | |
|---|---|
| Name | Compound of formula I-C |
| Concentration in the | 37 mg/ml (5 mL/kg, iv) |
| formulation | |
| Carrier | 0.9% NaCl |
| Required quantity | 1200 mg |
| Appearance | White powder |
| Storage conditions | Ambient temperature |
| Preparation protocol | Weigh the powder and dissolve it in the |
| | carrier. |
| Storage conditions for the | 4° C. |
| formulation | |
| Frequency of preparation | The day of the experiment |
| Shelf life of the formulation | 24 hours |

### 1.1.2. Reference Product

TABLE 4

| | |
|---|---|
| Name | Cariporide |
| Concentration | 12 mg/ml (5 mL/kg, iv) |
| Storage conditions | 4° C. |
| Preparation protocol | Solutions with 12 mg/ml in 100% DMSO were |
| | prepared and aliquoted. For each rat, one aliquot |
| | was used and dissolved in order to obtain a solution |
| | with 0.06 mg/ml in 0.5% DMSO. |
| Storage conditions | Solution with 12 mg/ml in 100% DMSO at −20° C. |
| for the formulation | Solution 0.06 mg/ml in 0.5% DMSO at ambient |
| | temperature. |
| Frequency of | Daily (for the dilution) |
| preparation | |
| Shelf life of | One day |
| the formulation | |

### 1.1.3. Carrier

Saline Solution

### 1.1.4. Animals Used

TABLE 5

| | |
|---|---|
| Species | Rat |
| Race | Sprague Dawley |
| Sex | Male |
| Age | / |
| Number | 36 animals have been included gradually as a function of |
| | the mortality rate (~30%) in order to obtain at least 8 |
| | evaluable animals per treatment group at the end of the |
| | experimental phase. |
| Weight | 250-300 g |
| Supplier | JANVIER SAS |
| Justification | Males were selected to avoid the hormonal cycle. |

The animals were housed in ventilated breeding cages which were GR900-enriched (905 CM², Tecniplast) throughout the entire acclimatisation period and the experimental phase. The animal cages were provided with sufficient nesting material to completely cover the animals (Sizzle-Nest: unbleached brown kraft paper from Bio-service), wooden sticks (aspen bricks from Bio-service). The animal cage bedding (particles of poplar wood, without chemical treatment, pre-comminuted, de-dusted, sieved and dehydrated popular wood, without chemical treatment, from SDS DIETEX) was changed at least once per week. They were housed in groups of 2 animals with a standard 12-hour light cycle (lights off at 20:00), at 22±2° C. and 55±10% relative humidity.

An acclimatisation period of at least 5 days was applied.

Throughout this phase, SDS water and tap water were supplied ad libitum.

Arrhythmias were obtained in an acute model of ischaemia-reperfusion in rats.

### 1.1.5. Treatment

Dosage Regimen and Test Groups:

TABLE 6

| Group | Nbr/ | | | | | Administration | Start of |
|---|---|---|---|---|---|---|---|
| No. | group | treatment | Route | Dose | | volume/rate | treatment |
| 1 | n = 12 | Carrier | IV | 0.9% | | Bolus | 30 minutes |
| | | (Saline | | | | 5 mL/kg | before |
| | | solution) | | | | | ischaemia |
| 2 | n = 11 | Compound | | 185 | mg/kg | | |
| | | I-C | | | | | |
| 3 | n = 9 | Cariporide | | 0.3 | mg/kg | | 5 minutes |
| | | | | | | | before |
| | | | | | | | ischaemia |

### 1.2. Method

### 1.2.1. Venous Catheterisation and Blood Pressure/ECG Recording

The rats were first anaesthetised, then intubated and mechanically ventilated, before being prepared for surgery.

Catheters were then placed in the carotid artery in order to measure the arterial pressure and in the caudal vein for the perfusion of drugs. The arterial catheter consists in a catheter filled with fluid which sends the pressure to a transducer located nearby. The mean arterial pressure is calculated electronically and recorded continuously.

A three-lead electrocardiogram (ECG) was recorded throughout the procedure by means of needle electrodes attached to the limbs.

The modifications to the ECG, mean arterial pressure (MAP) and the heart rate were measured before and during the occlusion-reperfusion period. The definitions of the arrhythmias are based on those described in the Lambeth conventions (Walker and al., The Lambeth conventions: guidelines for the study of arrhythmias in ischaemia infarction, and reperfusion, Cardiovascular Research, 1988, 22(7), 447-455). The ectopic activity has been categorized as a single ventricular premature beat (VPB), a ventricular tachycardia (VT, four or more consecutive VPBs) or a ventricular fibrillation (VF, inability to distinguish individual QRS complexes and to measure the rate). The arterial pressure traces were referred to in order to confirm which type of ectopic activity was produced, in particular to distinguish between polymorphic VT and VF. When the first is produced, the arterial pressure is generally still pulsatile whereas with VF, the arterial pressure falls rapidly towards zero and is no longer pulsatile. The VF can be sustained or can spontaneously return to a normal sinusoidal rhythm in rats. In all the experiments, the incidences of TV and VF as well as the duration of the ventricular tachycardia were noted.

### 1.2.2. Arrhythmia Induced by Ischaemia-Reperfusion in Anaesthetised Rats

The heart was then exposed by a left intercostal thoracotomy. After opening the pericardium, a 6.0 silk stitch was placed around the left coronary artery on a polypropylene tube in order to form a collar. The hearts were subjected to an ischaemia for 7 minutes by tightening the suture 5 minutes after the treatment. Finally, the collar occluder was released in order to allow the reperfusion of the myocardial tissue for 10 minutes. At the end of the reperfusion period, the rats were euthanised by cervical dislocation while still anaesthetised.

### 1.2.3. Data Analysis

The means± the standard error on the mean are presented.

The statistical analysis was carried out using the GraphPad Prism 5 software. The Fisher test was used to analyse the differences between the incidences of arrhythmias, while the chi-squared test was used to compare the incidence of mortality. The Kruskall-Wallis test was used to analyse the differences between the duration of the tachycardia and the ventricular fibrillations and the difference in the number of ventricular fibrillations.

For all the tests, p<0.05 will be considered as significant.

### 2. Results and Discussion

The rats were treated by the intravenous route with a saline solution, a compound of formula I-C at 185 mg/kg (30 minutes before the ischaemia) or cariporide at 0.3 mg/kg (5 minutes before the ischaemia). The animals were subjected to a 7-minute ischaemia and a 10-minute reperfusion. Throughout the experimental phase, the arterial pressure and ECG profiles were recorded continuously. Blood was sampled 5 minutes after the end of the reperfusion and the heart was weighed.

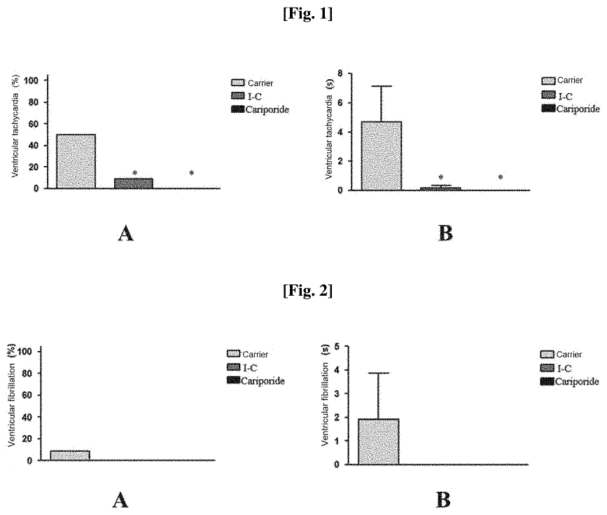

FIG. 1 shows the incidence (FIG. 1A) and duration (FIG. 1B) of the ventricular tachycardia during the ischaemia.

FIG. 2 shows the incidence (FIG. 2A) and duration (FIG. 2B) of the fibrillation, analysed during the ischaemia.

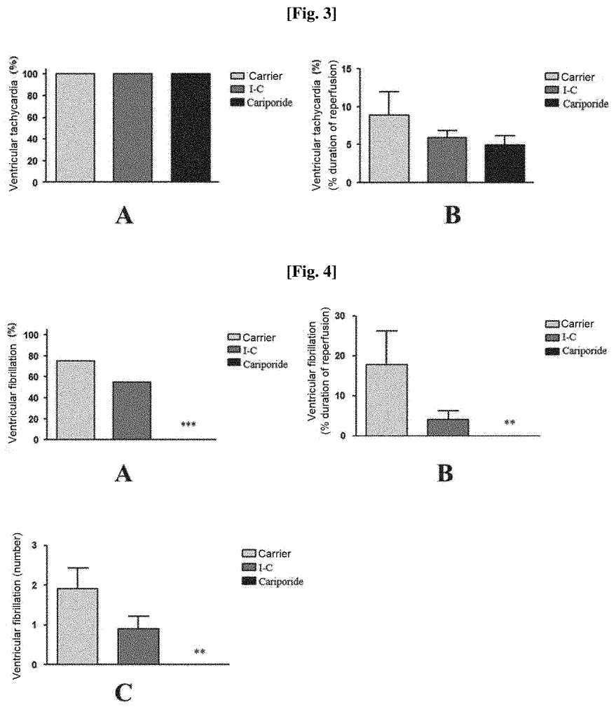

FIG. 3 shows the incidence (FIG. 3A) and duration (FIG. 3B) of the ventricular tachycardia during the reperfusion.

FIG. 4 shows the incidence (FIG. 4A) and duration (FIG. 4B) of fibrillation during the reperfusion, as well as the number of ventricular fibrillations (FIG. 4C) during this period.

In the group of the carrier, the ischaemia has caused a ventricular tachycardia in half of the animals (duration: 4.7±2.4 s) and a ventricular fibrillation was observed in one rat, whereas the reperfusion has caused a ventricular tachycardia in all the animals (duration: 8.9 t 3.1% of the duration of the reperfusion), and a ventricular fibrillation in 75% of the animals (duration: 17.8±8.4% of the reperfusion period with approximately two events per animal).

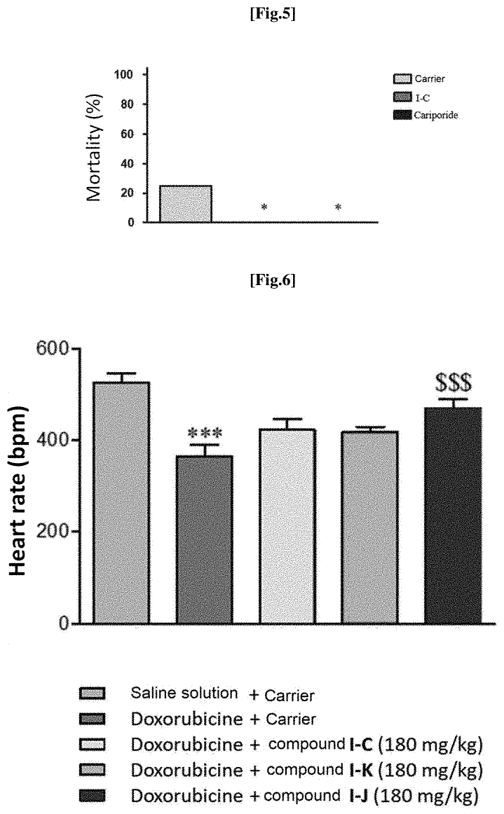

Moreover, as shown in FIG. 5, for 25% of the animals, the ventricular arrhythmias during reperfusion were irreversible and have led to death.

As envisaged, the preventative treatment with cariporide removed the ventricular tachycardia and fibrillation during the ischaemia. During reperfusion, cariporide has had no beneficial effect on the incidence of ventricular tachycardia, but the duration has had a tendency to be shorter with respect to the group of carriers. Cariporide has removed the ventricular fibrillation and mortality during the reperfusion period.

During the ischaemia, the preventative treatment by a compound of formula I-C has made it possible to significantly reduce the incidence and duration of ventricular tachycardia (incidence: approximately 9%, duration: 0.2±0.2 s) and to remove the ventricular fibrillation. As with cariporide, the compound of formula I-C has had no effect on the incidence of ventricular tachycardia during reperfusion, but has appeared to reduce the duration. Although it is not statistically significant, a tendency to reduce the incidence of ventricular fibrillation was observed during reperfusion in the group treated by the compound of formula I-C compared to the carrier (incidence: approximately 55% and duration: 4.0±2.3% of the duration of the reperfusion with approximately one event per animal). Finally and above all, the compound of formula I-C has removed mortality during the reperfusion period.

The weight of the heart was a similar between the groups.

### 3. Conclusion

Ischaemia-reperfusion has been used, previously for generating an infarction and studying the protective effect of Nicotinamide Mononucleotide (NMN) against this infarction (Journal of Cardiovascular Pharmacology and Therapeutics, 2019, pp. 1-11; J.Mol Cell Cardiol. 2018, Vol. 121, pp. 155-162).

In this model, the infarction is induced by an ischaemia of at least 30 minutes and a reperfusion of at least 60 minutes. These severe conditions make it possible to model the infarction, which is generally triggered by the obstruction of an artery which supplies the heart with blood and therefore with oxygen, causing death of the muscle cells of the heart over a more or less extended region. It has been shown that NMN protects the heart and enables the patient to recover better after the infarction.

Here, much less severe conditions, in other words a 7-minute ischaemia followed by a 10-minute reperfusion can induce a ventricular tachycardia and a (non-fatal and fatal) fibrillation. Cariporide has removed the arrhythmias during the ischaemic period, as well as the ventricular fibrillation and the mortality during the reperfusion. The compound of formula I-C has removed the tachycardia and the ventricular fibrillations during the ischaemic period, has had a tendency to prevent ventricular fibrillation during reperfusion and has removed mortality during the reperfusion.

### III. Evaluation of the Compounds of the Invention in a Model of Arrhythmia Induced by Doxorubicin

The goal of this study was to evaluate the effects of an i.p. administration of compounds I-C, I-J and I-K, at 180 mg/kg, on arrhythmias induced by doxorubicin.

### 1. Material and Methods

### 1.1.Materials

### 1.1.1. Animals

76 male mice, 8 weeks old on arrival, were obtained from Janvier Labs, Le Genest St Isle, 53941 St Berthevin, France. Each animal was identified with an electronic chip.

### 1.1.2. Products

The compounds I-C, I-J and I-K were tested and stored at +4° C. before use. The carrier was a physiological buffer.

### 1.2. Method

### 1.2.1. Preparation of Formulations

The powder of compounds I-C, I-J and I-K (180 mg/kg) was dissolved in the carrier (the solution was used at ambient temperature for a maximum of 1 day).

### 1.2.2. Arrhythmia Induced by Doxorubicin

Arrhythmias due to a cardiotoxicity were induced by a single intraperitoneal injection of doxorubicin (DOX) at 20 mg/kg. Doxorubicin was prepared at 2 mg/mL and the administration volume was 10 mL/kg. The control group received an injection of saline solution.

### 1.2.3. Experimental Groups

Description of the Groups:

Group 1: saline solution+Carrier

Group 2: Doxorubicin (20 mg/kg)+Carrier

Group 3: Doxorubicin (20 mg/kg)+Compound I-C (180 mg/kg)

Group 4: Doxorubicin (20 mg/kg)+Compound I-J (180 mg/kg)

Group 5: Doxorubicin (20 mg/kg)+Compound I-K (180 mg/kg)

Distribution of Groups:

Each group comprises 14 to 24 mice. As indicated in the regulations relating to non-clinical laboratory studies, the groups of test and control animals have been kept under identical conditions. The envisaged duration of the study was 11 days.

### 1.2.4. Induction with Doxorubicin

On Day 0, the mice received an administration of DOX (20 mg/kg) by the intraperitoneal route.

### 1.2.5. Treatment

The treatment with compounds I-C, I-J and I-K was initiated after 5 days before the injection of DOX, once per day from Day 5 to Day 0.

The mice were treated i.p with compounds I-C, I-J and I-K 30 minutes before the injection of DOX.

The mice were treated i.p with compounds I-C, I-J and I-K during the duration of the experiment (J0 to J5) once per day. The last injection took place 24 hours before the sacrifice.

### 1.2.6. Evaluation of Cardiac Function by Electrocardiogram

An electrocardiogram (ECG) was produced 5 days after the injection of doxorubicin in the anaesthetised animals (isoflurane 1.5-2%) by non-invasive, two-dimensional echocardiography (VF16-5 probe, Siemens, Acuson NX3 Elite).

In particular, the heart rate was evaluated during the ECG:

### 2. Results and Discussion

FIG. 6 shows the heart rate 5 days after injection of saline solution or DOX (20 mg/kg).

The doxorubicin considerably reduced the heart rate compared with the control mice (365.1±23.9 bpm vs 525.6±19.8 bpm respectively). The treatments with compounds I-C, I-J and I-K caused an increase in heart rates, the compound I-J significantly improving this parameter (470.1±18.8 bpm (p<0.0.001)).

Hence, the reduction in heart rate induced by doxorubicin was significantly attenuated by the treatment with compounds I-C, I-J and I-K.

1.-10. (canceled)

11. A method for treating arrythmia in a subject in need thereof, said method comprising administering to said subject a therapeutically effective amount of a compound of formula (I)× 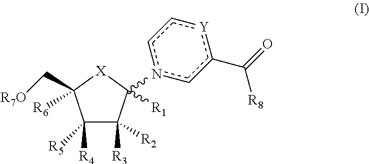
or a pharmaceutically acceptable salt and/or solvate thereof, wherein:
X is selected from O, CH₂, S, Se, CHF, CF₂ and C═CH₂;
R₁ is selected from H, azido, cyano, C₁-C₈ alkyl, C₁-C₈ thio-alkyl, C₁-C₈ heteroalkyl and OR; wherein R is selected from H and C₁-C₈ alkyl;
R₂, R₃, R₄ and R₅ are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein Ra is a side chain selected from a proteinogenic amino acid;
R₆ is selected from H, azido, cyano, C₁-C₈ alkyl, C₁-C₈ thio-alkyl, C₁-C₈ heteroalkyl and OR; wherein R is selected from H and C₁-C₈ alkyl;
R₇ is selected from P(O)R₉R₁₀, P(S)R₉R₁₀ and 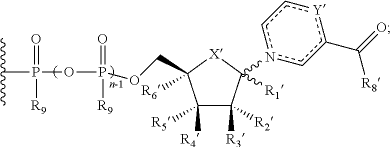
wherein
R₉ and R₁₀ are selected, independently of one another, from OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, (C₅-C₁₂)-aryl-(C₁-C₈)-alkyl,
(C₁-C₈)-alkyl-(C₅-C₁₂)-aryl, (C₁-C₈)-heteroalkyl, (C₃-C₈)-heterocycloalkyl, (C₅-C₁₂)-heteroaryl and NHCR_{α}R_{α′}C(O)R₁₂; wherein:
R₁₁ is selected from C₁-C₁₀ alkyl, C₃-C₁₀ cycloalkyl, C₅-C₁₂ aryl, (C₁-C₁₀)-alkyl-(C₅-C₁₂)-aryl, C₅-C₁₂ substituted aryl, C₁-C₁₀ heteroalkyl, C₁-C₁₀ haloalkyl, —(CH₂)ₘC(O)(C₁-C₁₅)-alkyl, —(CH₂)ₘOC(O)(C₁-C₁₅)-alkyl, —(CH₂)ₘOC(O)O(C₁-C₁₅)-alkyl, —(CH₂)ₘSC(O)(C₁-C₁₅)-alkyl, —(CH₂)ₘC(O)O(C₁-C₁₅)-alkyl, —(CH₂)ₘC(O)O(C₁-C₁₅)-alkyl-aryl; wherein m is an integer selected from 1 to 8; and P(O)(OH)OP(O)(OH)₂; an internal or external counter-ion;
R₁₂ is selected from hydrogen, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₂ aryl, (C₁-C₄)-alkyl-(C₅-C₁₂)-aryl and C₅-C₁₂ heteroaryl; wherein said aryl or heteroaryl groups are optionally substituted by one or two groups selected from halogen, trifluoromethyl, C₁-C₆ alkyl, C₁-C₆ alkoxy and cyano;
R₁₃ and R₁₄ are selected independently from H, C₁-C₈ alkyl and (C₁-C₈)-alkyl-(C₅-C₁₂)-aryl;
R_{α} and R_{α′} selected independently, from hydrogen, C₁-C₁₀ alkyl, C₂-C₁₀ alkenyl, C₂-C₁₀ alkynyl, C₃-C₁₀ cycloalkyl, C₁-C₁₀ thio-alkyl, C₁-C₁₀ hydroxylalkyl, (C₁-C₁₀)-alkyl-(C₅-C₁₂)-aryl, C₅-C₁₂ aryl, —(CH₂)₃NHC(═NH)NH₂, (1H-indol-3-yl)-methyl, (1H-imidazol-4-yl)-methyl and a side chain selected from a proteinogenic or non-proteinogenic amino acid; wherein said aryl groups are optionally substituted by a group selected from hydroxyl, C₁-C₁₀ alkyl, C₁-C₆ alkoxy, halogen, nitro and cyano;
or R₉ and R₁₀, with the phosphorus atoms to which they are bonded, form a 6-member-ring, wherein —R₉-R₁₀— represents —CH₂—CH₂—CHR— or —O—CH₂—CH₂—CHR—O—; wherein R is selected from hydrogen, C₅-C₆ aryl and C₅-C₆ heteroaryl; wherein said aryl or heteroaryl groups are optionally substituted by one or two groups selected from halogen, trifluoromethyl, C₁-C₆ alkyl, C₁-C₆ alkoxy and cyano;
X′ is selected from O, CH₂, S, Se, CHF, CF₂ and C═CH₂;
R_{1′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{2′}, R_{3′}, R_{4′} and R_{5′} are selected, independently of one another, from H, halogen, azido, cyano, hydroxyl, C₁-C₁₂ alkyl, C₁-C₁₂ thio-alkyl, C₁-C₁₂ heteroalkyl, C₁-C₁₂ haloalkyl and OR; wherein R is selected from H, C₁-C₁₂ alkyl, C(O)(C₁-C₁₂)-alkyl, C(O)NH(C₁-C₁₂)-alkyl, C(O)O(C₁-C₁₂)-alkyl, C(O)-aryl, C(O)(C₁-C₁₂)-alkyl-(C₅-C₁₂)-aryl, C(O)NH(C₁-C₁₂)-alkyl-(C₅-C₁₂)-aryl, C(O)O(C₁-C₁₂)-alkyl-(C₅-C₁₂)-aryl and C(O)CHR_{AA}NH₂; wherein RA is a side chain selected from a proteinogenic amino acid;
R_{6′} is selected from H, azido, cyano, C₁-C₈ alkyl, C₁-C₈ thio-alkyl, C₁-C₈ heteroalkyl and OR; wherein R is selected from H and C₁-C₈ alkyl;
R_{8′} is selected from H, OR, NHR_{15′}, NR_{15′}R_{16′}, NH—NHR_{15′}, SH, CN, N₃ and halogen; wherein R_{15′} and R_{16′} are selected, independently of one another, from H, C₁-C₈ alkyl and C₁-C₈ alkyl-aryl;
Y′ is selected from CH, CH₂, C(CH₃)₂ and CCH₃;
n is an integer selected from 1 to 3; [symbol]
represents a single or a double bond according to Y′; and [symbol]
represents the alpha or beta anomer according to the position of R_{1′};
R₈ is selected from H, OR, NHR₁₅, NR₁₅R₁₆, NH—NHR₁₅, SH, CN, N₃ and halogen;
wherein R is selected from H and C₁-C₁₂, alkyl, and R₁₅ and R₁₆ are selected, independently of one another, from H, C₁-C₈ alkyl and C₁-C₈ alkyl-aryl and —CHR_{AA}CO₂H wherein R_{AA} is a side chain selected from a proteinogenic or non-proteinogenic amino acid;
Y is selected from CH, CH₂, C(CH₃)₂ and CCH₃; [symbol]
represents a single or a double bond according to Y; and [symbol]
represents the alpha or beta anomer according to the position of R₁.

12. The method according to claim 11, wherein X represents oxygen.

13. The method according to claim 11, wherein R₁ and R₆ each represent hydrogen.

14. The method according to claim 11, wherein R₂, R₃, R₄ and R₅ each represent, independently of one another, hydrogen or OH.

15. The method according to claim 11, wherein Y represents CH.

16. The method according to claim 11, wherein Y represents CH₂.

17. The method according to claim 11, wherein R₇ represents P(O)R₉R₁₀ or 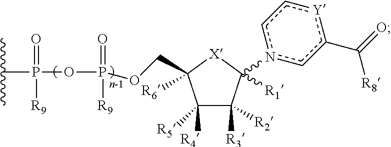
wherein R₉ and R₁₀ are as defined in claim 1 and
X′ is oxygen;
R_{1′} and R_{6′} each represent hydrogen;
R_{2′}, R_{3′}, R_{4′} and R_{5′} are independently selected from hydrogen and OH;
R_{8′} is NH₂;
Y′ is selected from CH and CH₂;
n is equal to 2; [symbol]
represents a single or a double bond according to Y′; and [symbol]
represents the alpha or beta anomer according to the position de R_{1′}.

18. The method according to claim 11, wherein R₇ represents P(O)(OH)₂.

19. The method according to claim 11, wherein the compound of formula (I) is selected from the list of compounds consisting of: 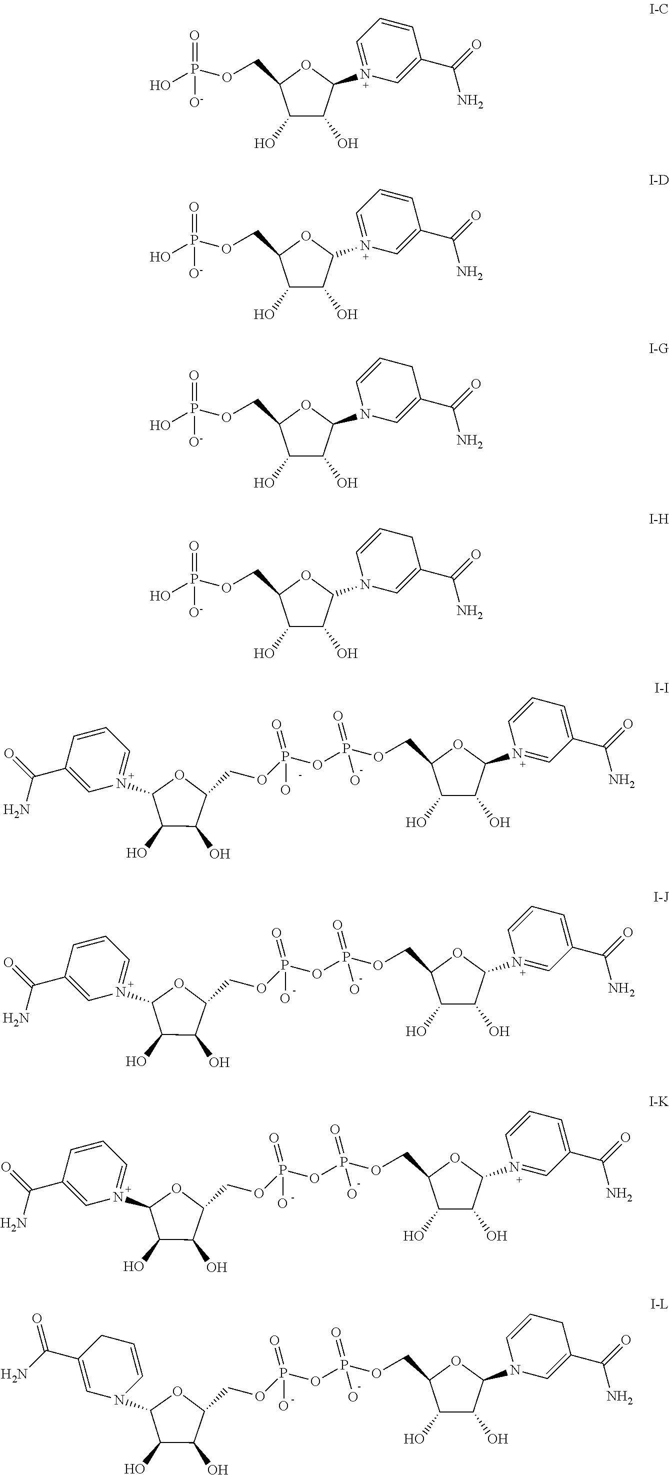 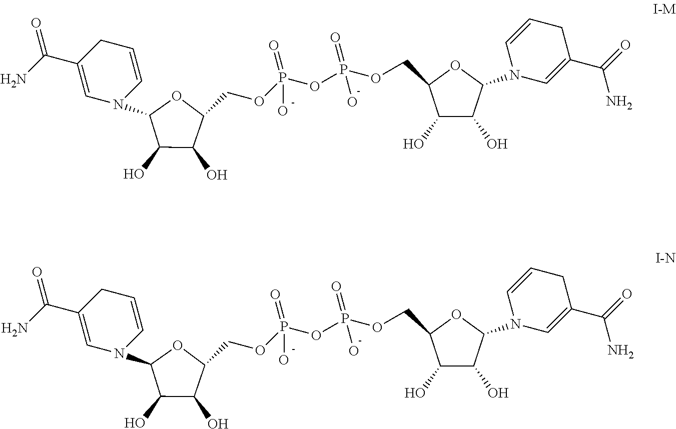 [SMILES: NC(=O)C1=CN([C@@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1 ; NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1]
and pharmaceutically acceptable salts and solvates thereof.

20. The method according to claim 11, wherein the type of arrhythmia is selected from the group consisting of bradycardia, tachycardia, auricular fibrillation, ventricular tachycardia and ventricular fibrillation.
